(19) United States
(12) Patent Application Publication
(10) Pub. No.: US 2015/0080753 A1
(43) Pub. Date: Mar. 19, 2015

(54) EVALUATION METHOD, EVALUATION DEVICE, PROGRAM, AND RECORDING MEDIUM

(71) Applicant: CANON KABUSHIKI KAISHA, Tokyo (JP)

(72) Inventors: Jungo Miyazaki, Kawasaki-shi (JP); Yoshikatsu Ichimura, Tokyo (JP)

(21) Appl. No.: 14/391,380

(22) PCT Filed: Apr. 8, 2013
(86) PCT No.: PCT/JP2013/002391

§ 371 Date: Oct. 8, 2014

(30) Foreign Application Priority Data
Apr. 12, 2012 (JP) 2012-091278
Dec. 13, 2012 (JP) 2012-272671

(51) Int. Cl. A61B 5/0484; G11B 27/031; G06F 19/00

(52) U.S. Cl. CPC A61B 5/04842; G06F 19/3406; G11B 27/031; A61B 5/04845
USPC 600/544; 386/278; 386/230

(57) ABSTRACT

An information acquiring method to acquire brain activity information from biological subjects presented a sensory stimulus, including the following processes,
(1) a process to present the sensory stimulus to at least one brain area of the biological subjects including the visual cortices of the cerebral cortex, the auditory cortices of the cerebral cortex, and the vestibular cortices of the cerebral cortex,
(2) a process to measure brain activity in multiple, different regions of the brain areas while the sensory stimulus is presented, and
(3) a process to evaluate stress provided to the subject by the sensory stimulus based on the brain activity data for the multiple regions.

### TECHNICAL FIELD

The present invention relates to an information acquiring method, information acquiring device, information acquiring program and recording medium for acquiring brain activity information from biological subjects presented a sensory stimulus.

### BACKGROUND ART

Watching video filmed on commercial video cameras with shakiness due to hand movement on a large screen is known to cause a feeling of discomfort similar to motion sickness in some viewers (so-called visually induced motion sickness). Also, watching 3D images is known to cause a feeling of comfort similar to visually induced motion sickness, which is called 3D motion sickness. The specific biological reactions caused by visually induced motion sickness and 3D motion sickness include nausea, dizziness, and disorientation.

According to “Cerebral blood flow and subjective score during visually induced motion sickness.”, Iijima, Atsuhiko, et al., 2008, 23rd BPES Symposium on Biological and Physiological Engineering, pp. 11-12 (hereinafter referred to as NPL 1), the method for evaluating visually induced motion sickness involves measuring changes in cerebral blood flow of viewers while watching video. Specifically, a 20-minute video which includes shakiness due to hand movement and swinging of the camera was presented to the subjects, and the changes in cerebral blood flows of the subjects was measured using a Near Infrared Spectroscopy and Imaging (NIRS) system (NIRO-200, Hamamatsu Photonics) wherein the relationship between the video and changes in cerebral blood flow was analyzed.

Also, the display system in Japanese Patent No. 3970759 (hereinafter abbreviated to PTL 1) discloses a method to instruct a display device with display conditions such as text, screen display size, resolution, display position, and such so that measurement data of biologically specific information such as brain waves produced by biological subjects, breathing, pulse, blood pressure, etc., and accumulated data in which the state of the user is accumulated are displayed in an optimum configuration.

The present inventors have uncovered a problem based on the method disclosed in NPL 1 and PTL 1. That is to say, according to the method in “Cerebral blood flow and subjective score during visually induced motion sickness.”, changes in cerebral blood flow were measured in the frontal cortex, which carry out executive functions such as reasoning and decision making. This makes it difficult to determine whether these changes in cerebral blood flow are due to the viewing of the video, or due to some other cause. For this reason, a method has been needed that could acquire information related to a stress similar to that of visually induced motion sickness so as to increase accuracy thereof.

Also, according to the method disclosed in PTL 1, methods and processes are established to accumulate biologically specific information on each individual user in order to acquire accurate values regarding the biologically specific information as it was unclear which biologically specific information indicated what kind of changes when the images are displayed. For this reason, a method has been needed that could acquire information related a stress similar to that of visually induced motion sickness so as to increase ease of use thereof.

### CITATION LIST

### Patent Literature

PTL 1: Japanese Patent No. 3970759

### Non Patent Literature

NPL 1: “Cerebral blood flow and subjective score during visually induced motion sickness.”, Iijima, Atsuhiko, et al., 2008, 23rd BPES Symposium on Biological and Physiological Engineering, pp. 11-12

### SUMMARY OF INVENTION

An evaluation method as in the present invention is an information acquiring method to acquire brain activity information from biological subjects presented with a sensory stimulus, the method including:

(1) a process to present the sensory stimulus to at least one brain area of the biological subjects including the visual cortices of the cerebral cortex, the auditory cortices of the cerebral cortex, and the vestibular cortices of the cerebral cortex

(2) a process to measure brain activity in multiple, different regions of the brain areas while the sensory stimulus is presented

(3) a process to acquire the brain activity information on the basis of the degree of similarity in brain activity in the different regions.

Also, an information acquiring device according to the present invention is to acquire brain activity information from biological subjects presented a sensory stimulus, including a stimulus presenting unit for presenting the sensory stimulus to at least one brain area of the biological subjects including the visual cortices of the cerebral cortex, the auditory cortices of the cerebral cortex, and the vestibular cortices of the cerebral cortex; a measuring unit to measure brain activity in the multiple, different regions of the brain areas; a calculating unit to calculate similarity in brain activity in the multiple regions of the brain areas; and a measuring control unit to control the measuring unit to measure the brain activity in the multiple regions of the brain areas while the sensory stimulus is presented by the stimulus presenting unit; wherein the brain activity information is acquired on the basis of the degree of similarity after measuring the brain activity in the multiple regions of the brain areas while the sensory stimulus is presented to the brain areas by the stimulus presenting unit.

Also, an information acquiring program according to the present invention is an information acquiring program to acquire brain activity information from biological subjects presented a sensory stimulus by executing the following processes:

(1) a process to present the sensory stimulus to at least one brain area of the biological subjects including the visual cortices of the cerebral cortex, the auditory cortices of the cerebral cortex, and the vestibular cortices of the cerebral cortex

(2) a process to measure brain activity in multiple, different regions of the brain areas while the sensory stimulus is presented

(3) a process to acquire the brain activity information on the basis of the degree of similarity in brain activity in the multiple different regions.

Also, a computer-readable recording medium according to the present invention records an information acquiring program in which the information acquiring program acquires brain activity information from biological subjects presented with a sensory stimulus by executing the following processes:

(1) a process to present the sensory stimulus to at least one brain area of the biological subjects including the visual cortices of the cerebral cortex, the auditory cortices of the cerebral cortex, and the vestibular cortices of the cerebral cortex

(2) a process to measure brain activity in multiple, different regions of the brain areas while the sensory stimulus is presented

(3) a process to acquire the brain activity information on the basis of the degree of similarity in brain activity in the multiple different regions.

Also, the information acquiring program according to the present invention is to execute processes to acquire brain activity information on the basis of the degree of similarity after measuring the brain activity in the multiple regions of the brain areas while the sensory stimulus is presented to the brain areas by the stimulus presenting unit using an information acquiring device which includes a stimulus presenting unit for presenting the sensory stimulus to at least one brain area of the biological subjects including the visual cortices of the cerebral cortex, the auditory cortices of the cerebral cortex, and the vestibular cortices of the cerebral cortex; a measuring unit to measure brain activity in the multiple, different regions of the brain areas; a calculating unit to calculate the similarity in brain activity in the multiple regions of the brain areas; and a measuring control unit to control the measuring unit to measure the brain activity in the multiple regions of the brain areas while the sensory stimulus is presented by the stimulus presenting unit.

Also, a computer-readable recording medium according to the present invention is to record an information acquiring program, wherein the information acquiring program executes processes to acquire brain activity information on the basis of the degree of similarity after measuring the brain activity in the multiple regions of the brain areas while the sensory stimulus is presented to the brain areas by the stimulus presenting unit using an information acquiring device which includes a stimulus presenting unit for presenting the sensory stimulus to at least one brain area of the biological subjects including the visual cortices of the cerebral cortex, the auditory cortices of the cerebral cortex, and the vestibular cortices of the cerebral cortex; a measuring unit to measure brain activity in the multiple, different regions of the brain areas; and a measuring control unit to control the measuring unit to measure the brain activity in the multiple regions of the brain areas while the sensory stimulus is presented by the stimulus presenting unit.

According to the present invention, a highly accurate method may be provided to acquire brain activity information from biological subjects presented a sensory stimulus. Also, a readily used method may be provided to edit and display the content of the sensory stimulus based on acquiring of the brain activity information.

Further features of the present invention will become apparent from the following description of exemplary embodiments with reference to the attached drawings.

### BRIEF DESCRIPTION OF DRAWINGS

FIG. 1 is a diagram describing an information acquiring method according to an embodiment of the present invention.

FIG. 2 includes diagrams describing the visual cortex.

FIG. 3 is a diagram illustrating the places to position electrodes according to the International 10-20 System.

FIG. 4 includes diagrams illustrating an expression for calculating a cross correlation coefficient.

FIG. 5 includes diagrams describing an example of calculating the cross correlation coefficient.

FIG. 6 is a diagram describing an information acquiring device according to an embodiment.

FIG. 7 is a diagram describing an example of an evaluation device according to an embodiment.

FIG. 8 is a diagram illustrating an fMRI device used in an embodiment.

FIG. 9A is an example of brain activity data acquired by an embodiment.

FIG. 9B illustrates time-series data for a cross correlation coefficient calculated from the brain activity data.

FIG. 10 is a table illustrating a cross correlation coefficient acquired by an embodiment.

FIG. 11A is a table illustrating cross correlation coefficients acquired by an embodiment.

FIG. 11B is a table illustrating cross correlation coefficients acquired by an embodiment.

FIG. 12A is a diagram illustrating an expression for calculating a cross correlation coefficient.

FIG. 12B is a diagram illustrating an expression for calculating a time-average value.

FIG. 12C is a diagram illustrating an expression for calculating an average rate of change.

FIG. 12D is a diagram illustrating an expression for calculating a mean-square coherence function.

FIG. 13 is a diagram describing an editing device according to an embodiment.

FIG. 14 is a diagram describing an example of the editing device according to an embodiment.

FIG. 15 is a diagram describing a display device according to an embodiment.

FIG. 16 is a diagram describing an example of the display device according to an embodiment.

FIG. 17 is a table illustrating the mean-square coherence function acquired by an embodiment.

### DESCRIPTION OF EMBODIMENTS

The following describes an embodiment of the present invention, but the present invention is not limited thusly.

The present inventors measured brain activity in the visual cortices of subjects during presentation of sensory stimuli (actual videos) by use of a functional magnetic resonance imaging (fMRI). The video consisted of two type of video, one video that caused little feeling of shakiness in the subjects, and one that caused a feeling of shakiness with a potential to also cause visually induced motion sickness. A subjective assessment was implemented to rate the stress level of subjects before and after the two videos were presented, and so confirmed that the stress score in the subjects was low after the presenting of the video with little feeling of shakiness, while the stress score in the subjects increased after the presenting of the video that has a potential to cause visually induced motion sickness. Brain activity data in the visual cortices during the presenting of the two videos was used to calculate a brain activity correlation in two different regions, and the brain activity correlation for the two video presented was tested for any statistically significant difference. As a result, the presenting of the video that has a potential to cause visually induced motion sickness was presented was confirmed to have a significant decrease in the brain activity correlation as compared to the presenting of the video that causes little feeling of shakiness. The brain activity correlation represents a cross correlation coefficient of the two sets of brain activity data, and quantifying how similar the temporal pattern of the two sets of brain activity data represents an example of a degree of similarity. Further, the difference in time-average value and the difference in the average rate of change of the brain activity data in the two regions were confirmed to be significantly larger when the video that has a potential to cause visually induced motion sickness was presented as compared to the when the video that causes little feeling of shakiness was presented. The difference in time-average values and the different in the average rate of change of the brain activity data for the two regions is an example of the degree of similarity.

Also, the present inventors acquired brain activity data from an electroencephalograph (EEG) using the same experimental procedure and video as the experiment in which the previously described fMRI was used. The measurement sites were two left and right posterior temporal regions near the visual cortices. The result of calculating a mean-square coherence function on the EEG data in these two left and right regions confirmed that the value of the mean-square coherence function decreased when presenting the video that has a potential to cause visually induced motion sickness as compared to when presenting the video that causes little feeling of shakiness. The aforementioned mean-square coherence function is an example of the aforementioned degree of similarity. The present invention has been made on the basis of the above experimental results. Details on the degree of similarity and the experimental data will be described later.

First, the information acquiring method according to the present embodiment will be described.

The information acquiring method according to the present embodiment is configured to acquire brain activity information from biological subjects presented with a sensory stimulus (for example, information related to stress), characterized by the inclusion of the following processes (1) through (3).

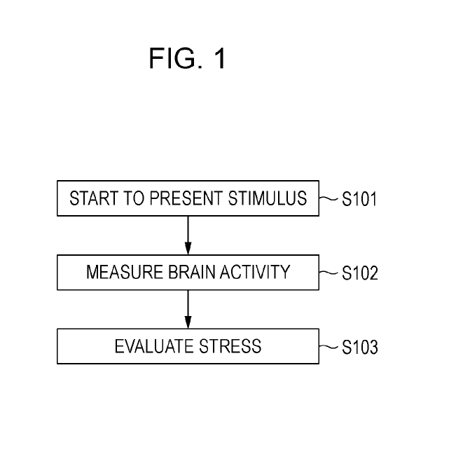

(1) As illustrated by step S101 in FIG. 1, this starts the presentation of a sensory stimulus to at least one region of the brain areas of the biological subjects including the visual cortices of the cerebral cortex (hereafter, the visual cortices), the auditory cortices of the cerebral cortex (hereafter, the auditory cortices), and the vestibular cortices of the cerebral cortex (hereafter, the vestibular cortices).

(2) As illustrated in step S102 in FIG. 1, this measures brain activity in multiple, different regions of the brain areas while the sensory stimulus is presented.

(3) As illustrated in step S103 in FIG. 1, this starts the acquirement of the brain activity information (for example, the degree of similarity) on the basis of brain activity data in the multiple regions.

### Process (1)

A sensory stimulus is presented to any of the brain areas of biological subjects (humans, for example) including the visual cortices such as by presenting a video, the auditory cortices such as by presenting a sound, and the vestibular cortices such as by applying acceleration.

### Process (2)

Brain activity for multiple, different regions of the brain areas is measured while presenting the sensory stimulus in process (1). For example, while presenting a video during process (1), neurons in the visual cortices of humans are activated. The brain activity may be quantified by measuring an action potentials in neurons in the brain, the magnetic field produced as a result thereof, or changes in cerebral blood flow.

Further, the measuring of brain activity while presenting a sensory stimulus means the acquisition of signals related to brain activity induced by presenting the sensory stimulus during a continuous or intermittent specific amount of time. In order to derive meaningful information from signals related to the brain activity, it is desirable to acquire signals over a specific amount of time, which is dependent on the type of brain activity and the type of device used to measure the brain activity. For example, when measuring brain activity as changes in the action potentials in neurons and changes in the magnetic field produced thereby, the neurons respond to the sensory stimulus on a time scale of a few milliseconds to a few hundred milliseconds, so it is desirable to use an electroencephalograph or magnetoencephalograph with sampling frequencies set to this time scale to detect signals related to brain activity for at least a duration of a few milliseconds to a few hundred milliseconds. Also, when measuring brain activity as changes in cerebral blood flow related to metabolic activity in the brain, these changes in cerebral blood flow respond on a time scale of a few seconds starting a few seconds later after the sensory stimulus has started, so it is desirable to use a NIRS device or an fMRI device with sampling frequencies set to this time scale to detect signals related to brain activity for at least a duration of a few seconds. As the time period to measure brain activity is increase, the amount of acquirable signals related to brain activity increases, and so the amount of information derived from these signals increases. On the other hand, however, the length of time in which the biological subject is restrained while their brain activity is measured also increases, which increases the risk of causing fatigue and introducing artifacts in the signals related to the acquired brain activity. Therefore, it is desirable to limit the time period for measuring brain activity from a few seconds to a few tens of minutes in order to establish a time period long enough to derive the desired information and not cause fatigue in the biological subjects whose brain activity is measured.

### Process (3)

Brain activity information is acquired on the basis of brain activity data for the multiple, different regions measured in process (2). Acquiring brain activity information may mean estimating the level of stress provided to biological subjects as an index of the correlation relationship between the brain activity data for the multiple, different regions, for example. When the correlation relationship between the brain activity data for the multiple regions is strong (degree of similarity is large), the stress provided to the subjects by the sensory stimulus may be evaluated as small, and when the correlation relationship is weak (degree of similarity is small), the stress provided may be evaluated as large. The correlation relationship (for example, level of similarity) may be quantified by using a cross correlation coefficient described later, or the like.

Further, according to the information acquiring method, stress changes over times may be comprehended by calculating a degree of similarity, which is one index related to stress from a cross correlation coefficient, etc. of brain activity data for the multiple, different regions, in sequential time series from the start of the sensory stimulus. That is to say, the specific points in time when stress significantly increases and decreases may be identified during a time period in which the sensory stimulus is presented to the biological subjects. Therefore, an editing method may be provided to edit the content of the sensory stimulus up to the point where the stress significantly increases so that the sensory stimulus does not provide an excessive stress to biological subjects, for example.

For example, when the sensory stimulus is a video, a display method may be provided to display video that has been adjusted to provide the optimum level of stress to biological subjects by including a process to display video edited by the editing method on a display device.

Using such a method to measure the brain activity of regions of brain areas which perform early processing of sensory information, i.e. the visual cortices, auditory cortices, and the vestibular cortices, rather than the brain areas such as the frontal cortex which governs executive functions, the brain activity information from biological subjects to which a sensory stimulus has been presented may be acquired, for example, such as information related to stress. For this reason, factors other than from the sensory stimulus is not likely to be present when acquiring information related to stress from the presentation of the sensory stimulus. Therefore, as factors other than the stimulus presented by the sensory stimulus to the subjects are not likely to have any influence, the information acquiring method according to the present embodiment may be said to be highly accurate as a method for acquiring information, for example, as an acquiring method for stress from biological subjects to which a sensory stimulus has been presented.

As only the brain activity in brain regions previously specified such as the visual cortices, auditory cortices, and vestibular cortices have to measure in response to various sensory stimuli such as visual stimuli, audio stimuli, and sense of balance stimuli, editing method and display method using the information acquiring method, the editing method and display method using the information acquiring method may be implemented using a device system with a minimum configuration of sensors for detecting brain activity data, and brain activity data also does not have to be accumulated on each individual user in advance, which results in a convenient display method and editing method of the content of the sensory stimulus such as video using the information acquiring method.

The information acquiring method, editing method and the display method using the information acquiring method according to the present embodiment is preferred when the sensory stimulus is a video. That is to say, brain activity information (for example, information related to stress) from biological subjects to which video is presented is acquired, based on the degree of similarity of brain activity in the visual cortices or vestibular cortices, and this video may be evaluated from the acquired brain activity information, edited, and the edited video may be displayed. Therefore, this method may be said to be highly accurate and convenient as a video evaluation method, video editing method, and video display method.

### Sensory Stimulus

The sensory stimulus in the present embodiment is not particularly restricted as long as it causes changes in brain activity for any of the brain areas of subjects including the visual cortices, auditory cortices, and the vestibular cortices. Examples include a visual stimulus, such as a video, that changes brain activity in the visual cortex, an auditory stimulus, such as sound, that changes brain activity in the auditory cortex, an audio-visual stimulus, such as a video with sound, that changes brain activity in the visual cortex and the auditory cortex, and a sense of balance stimulus, such as the feeling of acceleration provided to subjects by a roller coaster, that changes brain activity in the vestibular cortex. The sensory stimulus may be any one of the visual stimulus, auditory stimulus, or the sense of balance stimulus, or may be combination of a multiple of these stimuli. A flight simulator, for example, simultaneously presents visual stimulus, auditory stimulus, and a sense of balance stimulus to a biological subject. When the sensory stimulus is a video, the evaluation method, editing method, and display method according to the present embodiment are preferably an evaluation method, editing method, and display method of video.

### Biological Subjects

The biological subjects in the present embodiment indicate living organisms such as humans, monkeys, and dogs. Humans are the preferred subjects with respect to the information acquiring method according to the present embodiment. The following descriptions assume the biological subject to be human. Also, humans are sometimes referred to as subjects or viewers in the present specification.

### Brain Activity Information

The brain activity in the present embodiment represents a brain responses from biological subjects to which the previously described sensory stimulus is presented. The brain response may be quantified by measuring changes in action potentials in neurons in the brain, changes in the electromagnetic field produced by these changes in the action potentials, and changes in cerebral blood flow. The changes in cerebral blood flow are produced in accompaniment with the changes in electromagnetic fields, which are produced by the changes in the action potentials in the neurons, and they are also produced by other metabolic activity causes related to the cerebral circulation system. The action potentials in neurons in the brain may be measured using a single neuron recording device that uses electrodes. The changes in the electromagnetic field produced by changes in the action potentials may be measured by an electroencephalograph or a magnetoencephalograph. The changes in cerebral blood flow produced by changes in the action potentials may be measured using a functional magnetic resonance imaging (fMRI) device, a NIRS device, or a positron emission tomography device. Among these, a method using an electroencephalograph or a method using a NIRS device is preferable due to the low cost of the device and low restriction of subjects.

The brain activity information in the present embodiment represents information measurement data of the previously described brain activity and the information acquired from this data. For example, the brain activity information may be information related to stress. The information related to stress may be quantified using the degree of similarity between brain activity of multiple, different brain regions.

### Information Related to Stress

The stress in the present embodiment represents functional changes in the mind and body of subjects when various external stimuli work as strain on subjects. The subjects may exhibit such physiological responses as tiredness in the eyes, headache, sweating, nausea, dizziness, disorientation, eye strain, abnormalities of the autonomic nervous system, and abnormalities of the sense of balance system.

Another aspect of the stress according to the present embodiment represents functional changes in the mind and body when the various external stimuli work as a benefit on subjects. The external stimulus may be, for example, the presenting of a video with visual motion information on global scale such as video shot by a first-person perspective on a large screen, which produces a feeling of self-motion such that the subject feels as though they have moved through the space within the video (vection). Such a video may be used as a stimulus benefitting the feeling of presence, which produces a moderate stress in which the subject feels emotionally moved or a pleasurable experience. Whether the stress works as a strain or as a benefit to the subjects depends on the degree of the external stimulus and the susceptibility of the subjects, but the stress according to the present embodiment includes both of these aspects.

The information related to stress according to the present embodiment is information related to the level of stress and amount of changes thereof. The information acquiring method, editing method, and display method according to the present embodiment determines whether the level of stress and amount of changes thereof in the subjects to which the sensory stimulus was presented is within a predetermined range, and evaluates the sensory stimulus on the basis of this determination result, edits and then presents the edited sensory stimulus.

The information acquiring method according to the present embodiment preferably acquires information related to stress in biological subjects. Also, the information acquiring method, editing method, and display method, according to the present embodiment, may preferably be used when evaluating such stress causing biological responses such as visually induced motion sickness or 3D motion sickness, and editing and displaying the video contents.

### Visual Cortices

The sense of vision in the present embodiment is the sense in which visible light is physically input, and visual information refers to information regarding color, shape, motion, texture, and depth of physical objects in the outer world, information regarding categories of physical objects, and spatial information regarding positional relationship between physical objects. The brain area handling the early processing of visual information is the group of visual cortices.

When the brain activity to be measured is that in the visual cortices using the information acquiring method, editing method using this method, and the display method, in the present embodiment, any region of the brain areas exhibiting brain activity may be measured from among those in the visual cortices including the primary visual cortex (V1 (dorsal V1: V1d, ventral V1: V1v)), the secondary visual cortex (V2 (dorsal V2: V2d, ventral V2: V2v), the tertiary visual cortex (V3, V3 accessory: V3A), the fourth visual cortex (V4 (dorsal V4: V4d, ventral V4: V4v)), the middle temporal area (MT), the medial superior temporal area (MST), the seventh visual cortex (V7), the ventral posterior area (VP), the lateral occipital complex (LOc), and the eighth visual cortex (V8). Also, brain activity of regions spanning multiple brain areas (the region spanning V1 and V2, for example) in the visual cortices may be measured, or the brain activity in a region that is a portion of a brain area may be measured.

Further, there are theories stating other types of visual cortices equivalent to the middle temporal area (MT) and the medial superior temporal area (MST), and other regions that respond to visual motion near these visual cortices which are collectively labeled as the middle temporal complex (MT+). The present invention is governed by the classification method disclosed in “Retinotopy and functional subdivision of human areas MT and MST”, Huk, A. C., et al., The Journal of Neuroscience, 22: 7195-7205 (2002), which is currently the most widely accepted definition, but the present invention is not limited thusly. The brain areas specified include visual cortices which have a potential to be identified near these areas in the future, and are labeled here as the middle temporal area (MT) and the medial superior temporal area (MST) for convenience. In addition, there are theories stating other types of visual cortices equivalent to V4v and V8, and other types labeled V01 and V02 according to the present invention, these brain areas are specified with the labels V4v and V8. Among these regions, measuring the V3A, middle temporal area (MT), and the middle superior temporal area (MST), which process information relating to visual motion or binocular disparity, is preferable as this is thought to enable precise evaluation of stress causing biological responses such as visually induced motion sickness and 3D motion sickness. In the middle temporal area (MT), there are many neurons which selectively respond to objects moving in the visual field in specific direction or at specific speeds. For this reason, it is preferable to reference the neural response in the middle temporal area (MT) in order to detect visually induced motion sickness caused by stress from visual motion.

Visual information is processed in the visual cortices, which are confirmed to be in the occipital lobe, a portion of the parietal lobe, and a portion of the temporal lobe. Further, the frontal cortex is positioned in the anterior of the cerebral cortex, the occipital lobe is positioned in the posterior, the parietal lobe is positioned at the superior, and the temporal lobe is positioned on the lateral.

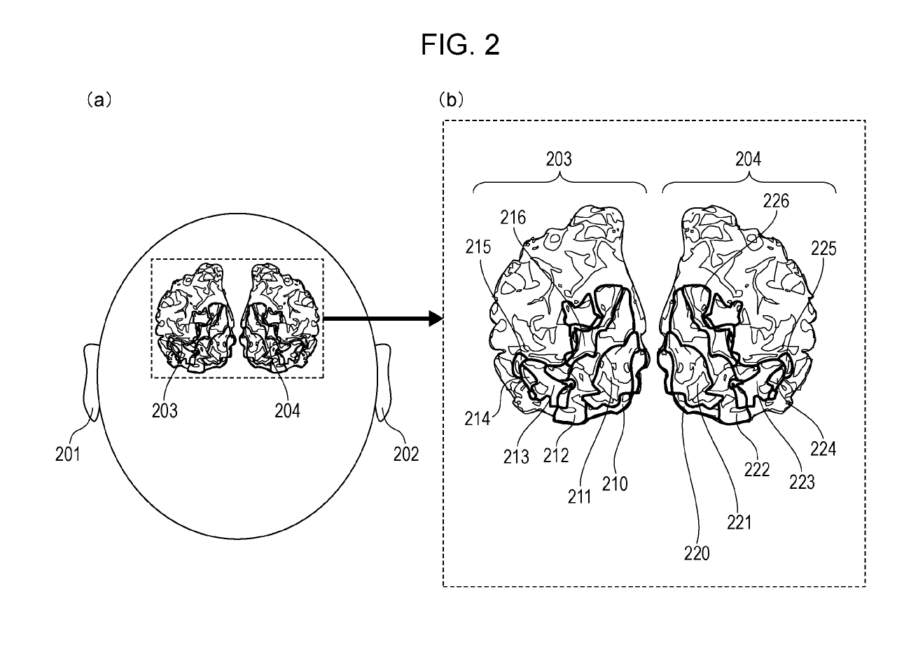

FIG. 2(a) is a schematic diagram of the surface of the cerebral cortex looked at from the occipital side. FIG. 2(b) is a magnified view of the surface of the cerebral cortex in FIG. 2(a). FIGS. 2(a) and 2(b) illustrate the cerebrum divided into left and right hemispheres for descriptive purposes, though the cerebrum is actually connected by the corpus callosum.

FIG. 2(a) illustrates a left ear 201, a right ear 202(a) left cerebral hemisphere 203, and a right cerebral hemisphere 204. A V1 (V1v, V1d) 210, V2 (V2v, V2d) 211, V3 212, lateral occipital complex (LOc) 213, middle temporal area (MT) 214, medial superior temporal area (MST) 215, and V3A 216 are illustrated in the left cerebral hemisphere 203 (FIG. 2(b)). Also, a V1 (V1v, V1d) 220, V2 (V2v, V2d) 221, V3 222, lateral occipital complex (LOc) 223, middle temporal area (MT) 224, medial superior temporal area (MST) 225, and V3A 226 are illustrated in the right cerebral hemisphere 204 (FIG. 2(b)).

As illustrated in FIG. 2(b), each visual cortex is positioned nearly symmetrically in both hemispheres. The visual information is first input toward the V1 in the posterior end of the occipital lobe in both the left and right hemispheres. Afterwards, information processing is performed in the visual cortices that contribute to a higher-order function such as V2, V3, and V3A. The visual information processed in the occipital lobe visual cortices travels up through each visually related cortex of the parietal lobe, the temporal lobe, and the frontal lobe, where it is integrated to form such visual functions as visual perception and visual memory. Further, V1 is positioned at area 17, V2 is positioned at area 18, and V3 is positioned at area 19 on Brodmann's brain map.

Visual information processing after V1 and V2 are primarily processed in two paths within the visual cortices. The first is called the dorsal stream, and this visual information processing pathway goes through brain areas that extend into the posterior side of the cerebral cortex such as V3, V3A, the middle temporal area (MT), the medial superior temporal area (MST), and the lateral occipital complex (LOc) V7. Many neurons that respond to visual motion and binocular disparity exist in the dorsal stream, and are thought to be involved in perception of one's position in space and motive state. The other is called the ventral stream, and this visual information processing pathway goes through brain areas that extend into ventral part of the cerebral cortex such as ventral fourth visual cortex (V4v) and the eighth visual cortex (V8). Many neurons that respond to color, shape, or the categories of objects exist in the ventral stream, and are thought to be involved in recognition of visual object representation.

When visually induced motion sickness occurs, neural responses in the visual cortices to the visual stimulus are entangled. That is to say, for the state in which stress caused by the video is presented to the viewer, the temporal pattern of brain activity in each brain area is entangled, and the brain activity correlation between the visual cortices decreases significantly. Therefore, information related to stress such as visually induced motion sickness and 3D motion sickness may be acquired by indexing the degree of similarity quantifying how similar the temporal patterns are regarding brain activity in the visual cortices.

Further, the decrease in brain activity also causes changes in metabolic activity of the cerebral circulation system, which is represented by the amount of blood flow in the visual cortices and the difference in changes thereof. For example, as visually induced motion sickness progresses, the amount of blood flow and the amount of change thereof increases on one side of the middle temporal area (MT), and the amount of blood flow and the amount of change thereof decreases on the other side of the middle temporal area (MT). Therefore, information related to stress such as visually induced motion sickness and 3D motion sickness may be acquired by indexing the degree of similarity quantifying how similar the changes over time are regarding the amount of blood flow and the amount of change thereof in the visual cortices.

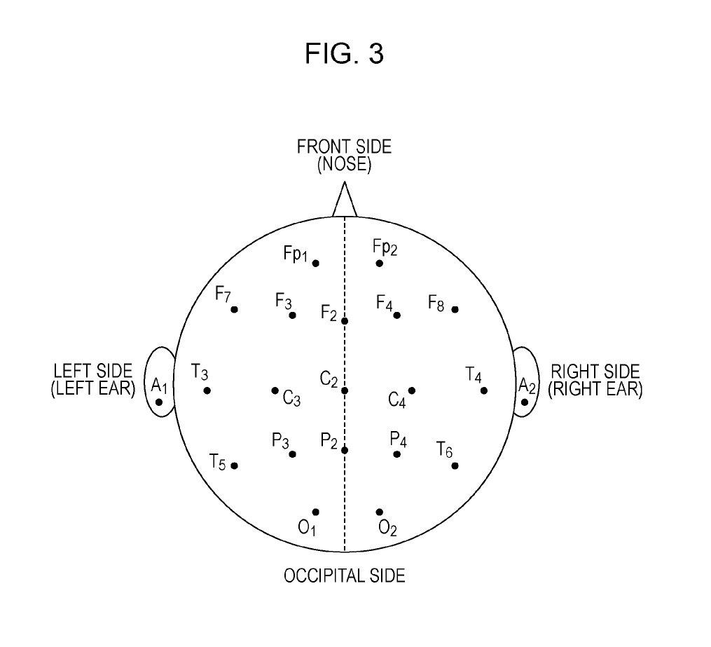

When measuring activity in the visual cortices with an electroencephalograph (EEG), the brain areas distributed at the entrance to the visual cortices such as V1, V2 and V3 are in the posterior side of the occipital lobe, and so signals may be detected from electrodes O₁ and O₂ as in the International 10-20 System. Also, the brain areas V3A and V7 are distributed at the parietal lobe side, and so it is thought that signals may be detected from electrodes P₂, P₃, and P₄ as in the International 10-20 System, and V4d, the middle temporal area (MT); the medial superior temporal area (MST), and the lateral occipital complex (LOc) are distributed at the temporal lobe side, so signal may be detected from electrodes T₅ and T₆ as in the International 10-20 System. Note however, that brain waves spread spatially, so the electrodes used to detect signals from the visual cortices are not limited to those described above. The International 10-20 System is an international reference system that determines where to position measuring electrodes on the head when measuring brain activity by an electroencephalograph. FIG. 3 is a diagram illustrating places to position electrodes according to the International 10-20 System. For example, measuring brain activity for V1, V2, V3, etc. with an electroencephalograph may be performed by placing electrodes in positions O₁ and O₂ as in FIG. 3. Further, though it is preferable to determine electrode positions using the International 10-20 System, but electrode positions are not particular restricted.

According to the evaluation method, editing method, and display method according to the present embodiment, when the sensory stimulus is a video and the viewing of this video has a potential to cause 3D motion sickness in the subject, it is preferable to measure brain activity in at least one region of the brain areas for the visual cortices likely to show the influence of 3D motion sickness, that is to say, the visual cortices in the dorsal stream in which many neurons exist that selectively respond to binocular disparity including V3, V3A, V7, the middle temporal area (MT), the medial superior temporal area (MST), and the lateral occipital complex (LOc). These brain areas are distributed from the occipital lobe to the parietal lobe, and so information related to stress in viewers caused by 3D video may be accurately and readily acquired by referencing brain activity time-series data detected by electrodes O₁ and O₂ in the left and right occipital areas as in FIG. 2 and by electrodes P₂, P₃, and P₄ in the left and right parietal areas. After performing this kind of brain activity measurement, the degree of similarity such as a cross correlation coefficient described later is calculated using the brain activity time-series data detected by the electrodes O₁ and O₂ afterwards, this degree of similarity is compared with a previously set threshold to determine whether the video under evaluation provided stress such as 3D motion sickness to the viewer. The calculation method of the degree of similarity such as the cross correlation coefficient and the setting method of the threshold may be performed by the methods described later.

### Sense of Hearing, Auditory Cortices

The sense of hearing in the present embodiment is the sense in which sound is physical input, and audio information refers to strength of sound, pitch, timbre, sound source direction, rhythm, and pronunciation. The brain areas related to the sense of hearing are the auditory cortices. The auditory cortices include the primary, secondary, and tertiary auditory cortices, and are positioned in area 41 and area 42 on Brodmann's brain map. When measuring brain activity for the auditory cortices by an electroencephalograph, signals may be detected by electrodes T₃ and T₄ as in the International 10-20 System. However, brain waves spread spatially, and so the electrodes used to detect signals from the auditory cortices are not limited to T₃ and T₄.

### Sense of Balance, Vestibular Cortices

The sense of balance in the present embodiment is the sense in which information regarding the sense of balance (information on how far a person is leaning, moving or not, etc.) is physically input. The brain area related to the sense of balance is the vestibular cortices including the parieto-insular vestibular cortex (PIVC). According to the present embodiment, the vestibular cortices are defined as being positioned near the temporo-parietal junction, which is where the temporal and parietal lobes meet at the posterior end of the Sylvian fissure. When measuring brain activity for the vestibular cortices by an electroencephalograph, it is thought that signals may be detected by electrodes T₃, T₄, P₃, and P₄ as in the International 10-20 System. However, brain waves spread spatially, and so the electrodes used to detect signals from the vestibular cortices are not limited to T₃, T₄, P₃, and P₄.

The following describes an example using the visual cortices, but the auditory cortices and the vestibular cortices may also be measured in the same way.

### Multiple, Different Regions

The multiple regions for measuring brain activity regarding the information acquiring method, the editing method and the display method using the information acquiring method according to the present embodiment may be selected freely from at least any region of the brain areas including the previously described visual cortices, auditory cortices, and the vestibular cortices. For example, two different brain areas within the visual cortices may be selected (e.g., V1 and V2), or a region spanning multiple brain areas within the visual cortices and specific brain areas within the visual cortices may be selected (e.g., the region spanning V1 and V2 and V3). Also, a region spanning multiple brain areas within the visual cortices and another region spanning multiple brain areas within the visual cortices brain areas may be selected (e.g., the region spanning V1 and V2 and the region spanning the middle temporal area (MT) and the medial superior temporal area (MST)). Also, when measuring brain activity a particular region, the brain activity in a portion of the region may be measured (e.g., a partial region of V1). Further, when measuring brain activity in regions spanning multiple brain areas, the brain region in a portion of the multiple brain areas may be measured, (e.g., a region of a portion of V1 and a region in a portion of V2). Though this description used the visual cortices as examples, the auditory cortices and the vestibular cortices are handled in the same way.

Also, the multiple, different regions may be selected from the visual cortices, or at least two brain areas from the visual cortices, auditory cortices, and the vestibular cortices may be selected, selecting one region from each brain area. For example, one region from the visual cortices, and one region from the vestibular cortices may be selected.

For example, when the previously described sensory stimulus is a video, information related to stress provided to the viewer by the video may be acquired by calculating the degree of similarity between the brain activity time-series data for one region in the visual cortex in the left hemisphere of the viewer who is presented the video and the brain activity time-series data for one region in the visual cortex in the right hemisphere. Particularly, when determining whether the video to be evaluated (hereafter, the video under evaluation) causes stress such as visually induced motion sickness or 3D motion sickness, or the degree of the stress, or when editing and displaying the video under evaluation based on the results of the determination, it is preferable to use brain activity time-series data in regions in the dorsal stream in the left and right hemispheres likely to show the influence of visual motion in the video under evaluation. Specifically, when determining whether the video causes visually induced motion sickness in the viewer, or the degree of the stress caused by visually induced motion sickness, or when editing and displaying the video under evaluation based on the results of the determination, it is preferable to index the degree of similarity of the brain activity between the middle temporal area (MT) in the left and right hemispheres and the medial superior temporal area (MST) in the left and right hemispheres in which many neurons exist that selectively respond to visual motion. Also, when determining whether the video causes 3D motion sickness in the viewer, or the degree of the stress caused by 3D motion sickness, or when editing and displaying the video under evaluation based on the results of the determination, it is preferable to index the degree of similarity of the brain activity between the tertiary visual cortices such as V3 and V3A, and the lateral occipital complex (LOc) in the left and right hemispheres in which many neurons exist that selectively respond to binocular disparity.

The degree of similarity such as the brain activity correlation between other multiple, different regions in the left hemisphere and the right hemisphere may be indexed. In this case, it is preferable to use brain activity time-series data for regions in the dorsal stream in the left and right hemispheres likely to show the influence caused by visual motion of the video or binocular disparity in order to determine whether the video under evaluation causes stress such as visually induced motion sickness or 3D motion sickness. For example, the degree of similarity for the brain activity of the primary visual cortex V1 and the middle temporal area (MT) in the left hemisphere may be indexed. Similarly, the degree of similarity for the brain activity of the primary visual cortex V1 and the middle temporal area (MT) in the right hemisphere may be indexed.

Also, neurons that selectively respond to visual motion or binocular disparity also exist in the ventral stream. Therefore, the calculation of the degree of similarity is not limited to the regions with the dorsal stream, thus brain activity time-series data for multiple, different regions in the visual cortices including the ventral stream may be used.

### Brain Activity Measuring Method

Details on devices to measure brain activity will now be described.

### Electroencephalograph

The electroencephalograph in the present embodiment measures electromagnetic fields produced by the action potentials in neurons in the brain. Active electrodes are attached to the head of a subject, and time-series data between a reference electrode with an electrical potential of zero (normally an electrode attached to the earlobe) and changes in the electrical potential is measured.

Measuring brain waves are performed when the previously described sensory stimulus is presented.

Brain wave measurement is preferably performed placing the electrodes in compliance with the International 10-20 System. FIG. 3 is a schematic diagram illustrating the electrode arrangement for the electroencephalograph. The diagram is from the perspective of the top of the head of a viewer 1, in which the upper half of FIG. 3 is the front (nose), and the lower half of FIG. 3 is the back (occipital side), the left half of FIG. 3 is the left side (left ear), and the right half of FIG. 3 is the right side (right ear). A₁, A₂, O₁, O₂, P₂, P₃, P₄, C₂, C₃, C₄, F₂, F₃, F₄, T₃, T₄, T₅, T₆, F₂, F₃, F₄, F₇, F₈, Fp₁, and Fp₂ represent each electrode. A₁ and A₂ are called the reference electrodes, and are typically attached near the earlobe which has an equivalent electrical potential of zero. The other electrodes are called the active electrodes, and detect the changes in electrical potential as compared to the reference electrode as brain wave signals. O₁ and O₂ detect brain waves in the occipital region, P₂, P₃, and P₄ detect in the parietal region, C₂, C₃, and C₄ detect in the central region, T₃, T₄, T₅, and T₆ detect in the temporal region, and F₂, F₃, F₄, F₇, F₈, Fp₁, and Fp₂ detect in the frontal region.

Although the spatial resolution of the electroencephalograph is inferior to a functional magnetic resonance imaging device, the device configuration is simple and the position of those being measure is not restricted, which enables brain activity to be measured in a more natural viewing environment.

When using brain activity time-series data detected from various electrodes in the left and right occipital areas, noise from biological activity other than the brain activity such as myoelectric potential and sweating, and noise from the electroencephalograph such as adhesion failure of electrodes are included in the detected brain activity time-series data. Therefore, it is preferable to perform a filter processing to remove noise components other than the brain activity response to the sensory stimulus as a preprocessing.

### NIRS Device

The Near Infrared Spectroscopy (NIRS) device measures changes in cerebral blood flow. Similar to other organs, blood circulates in the brain to supply nutrients, oxygen, and substances necessary for tissue maintenance, and to remove waste products. Particularly, in addition to the homeostatic circulation action of cerebral blood flow, a hemodynamic status is also active in response to changes in the action potentials in neurons. The neurons consume oxygen during activation, and become temporarily oxygen-starved. In order to resolve this, blood is sent to blood vessels in the brain near these neurons to supply oxygen to the neurons. This is a phenomenon called hemodynamic response. The NIRS device is a brain activity measuring method that measures changes in the amount of cerebral blood flow and the ratio of oxygenated hemoglobin and deoxygenated hemoglobin included therein, occurring due to the aforementioned hemodynamic response and perpetual metabolism activity of the cerebral circulation system.

A light-sending and light-receiving probe is attached to the head of a subject when using the NIRS device. The light-sending probe illuminates near-infrared light toward the interior of the brain of the subject, and the near-infrared light is reflected back to the surface of the head by the cerebral cortex, which is detected by the light-receiving probe. The oxygenated hemoglobin and the deoxygenated hemoglobin included in the cerebral blood flow has different absorption spectrums in response to the light in the near-infrared wavelength region, the near-infrared light illuminated from the light-sending probe is absorbed by the oxygenated hemoglobin and the deoxygenated hemoglobin included in the cerebral blood flow, and so the reduction in the amount of light detected by the light-receiving probe reflects the amounts of oxygenated hemoglobin and the deoxygenated hemoglobin. Therefore, the amount of blood flow in the area of the brain to which the near-infrared light passed and the ratio between the oxygenated hemoglobin and the deoxygenated hemoglobin included therein may be estimated from the changes in the amount of light from when illuminated to when detected. By measuring the changes in the amount of light over time, the temporal changes in the amount of cerebral blood flow for the area to which the light was illuminated and the ratio between the oxygenated hemoglobin and the deoxygenated hemoglobin included therein may be recorded as brain activity time-series data.

fMRI Device

The fMRI device is a device to visualize changes in blood flow related to brain activity using an MRI device. When using an fMRI device to measure changes in cerebral blood flow, normally brain activity spatial patterns are visualized by identifying the area that responded to the sensory stimulus by statistical analysis, and overlaid on an anatomical image as a color map.

The device configuration and signal detection principle of the fMRI device is different to that of the NIRS device, but such points as measuring changes in cerebral blood flow and the ability to evaluate stress provided to subjects by the sensory stimulus are the same as that of the NIRS device, and so such description is omitted here. However, the transmitted near-infrared light may be absorbed by brain tissue or may become scattered, and so as there are limits to the distance of propagation through brain until the light is detected by the light-receiving probe, the NIRS device may only be used to measure brain activity of areas near the surface of the brain such as the cerebral cortex. In contrast, the fMRI device detects signals correlating to the ratio of oxygenated hemoglobin included in the cerebral blood flow using radio waves as the probe, so there are no negative effects such as absorption by brain tissue or scattering, and brain activity measurement is not limited to the surface of the brain but also includes deep areas of the brain. These points represent different aspects of two devices.

### Brain Activity Information Acquiring Method

The brain activity information, for example, information related to stress, is acquired by analyzing data acquired by measuring the previously described brain activity. The information acquiring method, the evaluation method using this method, the editing method, and the display method according to the present embodiment are implemented based on the acquirement of information related to stress.

### Stress Evaluation Method

The method to evaluate stress is performed based on brain activity data (for example, degree of similarity) for multiple brain regions in the subject while the sensory stimulus was presented. For example, the degree of similarity related to the temporal patterns in the brain activity data for two brain regions while the stress from the sensory stimulus was presented is indexed. The degree of similarity may be quantified by comparing the cross correlation coefficients, the difference in time-average values, or the difference in the average rates of change regarding two sets of brain activity data. Details on the different types of degrees of similarity including the cross correlation coefficient, coherence function, difference in time-average values, and the difference in the average rates of change will be described next.

### Cross Correlation Coefficient

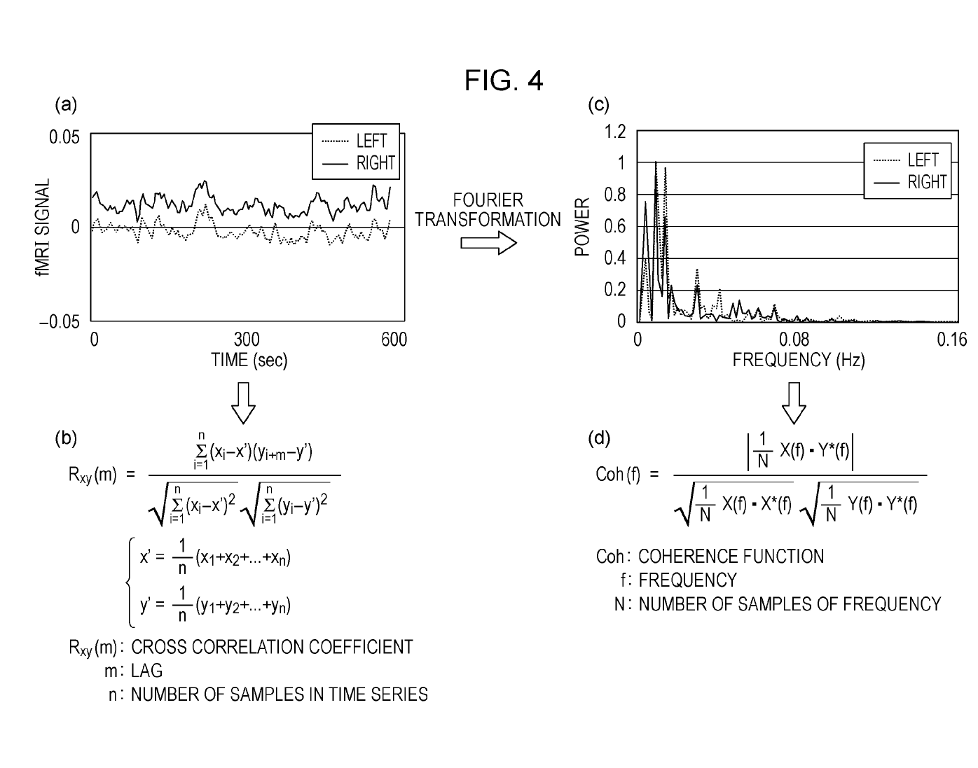

The cross correlation coefficient in the present embodiment is an index in which similarities in two sets of time-series data is quantified. For example, each set of the two sets of time-series data is vectored, the average value of all vectored elements is zero, and after normalizing the size of the vectors to produce a value of one, the inner product of these two vectors become the cross correlation coefficient. The cross correlation coefficient may be generally acquired by the expression illustrated in FIG. 4(b).

The method to acquire the cross correlation coefficient from time-series data (x₁, x₂, x₃, . . . , xₙ) of the brain activity regarding the information acquiring method according to the present embodiment is described below.

(i) Data is acquired by subtracting an average value x′ from all elements of the time-series data of the brain activity acquired by process (2) previously described (x₁, x₂, . . . , xₙ)

(ii) The square root of the sum of all elements acquired by (i) squared is calculated.

(iii) The value acquired by (ii) is divided by that of (i).

(iv) The same process from (i) to (iii) is executed on the time-series data (y₁, y₂, . . . , yₙ) of the brain activity acquired by process (2) previously described.

(v) The inner product of (iii) and (iv) is calculated.

The cross correlation coefficient acquired by performing the processes (i) through (v) is equivalent to performing a calculation of the expression illustrated in FIG. 4(b) or

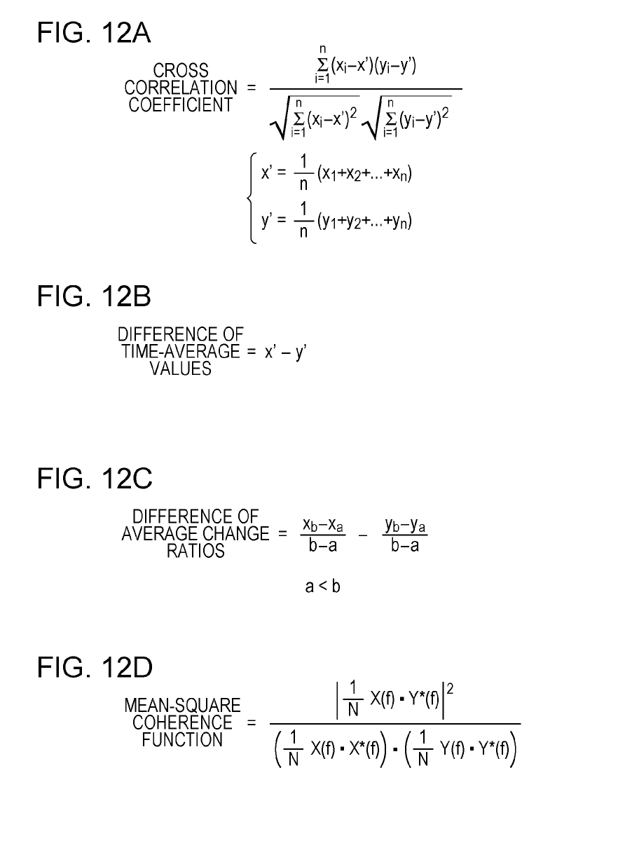

FIG. 12A by substituting the brain activity data values measured (x₁, x₂, . . . , xₙ) and (y₁, y₂, . . . , yₙ).

When the cross correlation coefficient is near one, the temporal pattern of the brain activity between the regions is not entangled, and the stress provided to the subjects by the sensory stimulus is small. Conversely, when the value is near zero, the temporal pattern of the brain activity between the regions is entangled, and the stress provided to the subjects is large.

By calculating the cross correlation coefficient in this way, information related to the stress provided to the subjects by the sensory stimulus may be acquired. Therefore, video is evaluated by the information acquiring method according to the present embodiment as such: when the sensory stimulus is a video, if the cross correlation coefficient is large, the video under evaluation does not provide stress or is not likely to provide stress; and when the cross correlation coefficient is small, the video under evaluation provides stress or is likely to provide stress.

The following is an example of such an evaluation. When using a video as the sensory stimulus, brain activity data for two different regions in the visual cortices are measured while the video is presented. The cross correlation coefficient is calculated using the brain activity data for the two regions and following steps (i) through (v) described previously. If there are multiple subjects or multiple measuring environments, the measurements are performed to acquire the differences in these cross correlation coefficients. Also, the average value of the acquired cross correlation coefficients, or the value of the smallest cross correlation is acquired as the threshold. When smaller than this threshold, it is evaluated as having stress or that the stress is large, and when larger than this threshold, it is evaluated as not having stress, or that the stress is small. The procedure to set the threshold is not limited to only the previously described method.

By calculating the cross correlation coefficient in this way, stress provided to subjects by the sensory stimulus may be evaluated.

FIG. 4 is a diagram illustrating the flow regarding the calculation method of the correlation value between the brain activity time-series data. The example in FIG. 4(a) illustrates brain activity time-series data from the primary visual cortex V1 in the right and left hemispheres detected by a functional magnetic resonance imaging device. In FIG. 4(a), the dotted line represents the brain activity time-series data of V1 in the left hemisphere, and the solid line of V1 in the right hemisphere. When calculating the cross correlation coefficient, which is the correlation value in the temporal regions, the brain activity time-series data from V1 in the right and left hemispheres is substituted into the expression for calculating the cross correlation coefficient defined as in FIG. 4(b). Here, x₁ and yᵢ (i=1, 2, . . . , n) are the time-series data, and R_{xy} (m) represents the cross correlation coefficient of xᵢ and yᵢ. m represents the lag in the two sets of time-series data, which is calculated as zero.

### Coherence Function

The cross correlation coefficient is an index quantifying the degree of similarity between two signals for a given time domain, and conversely, the coherence function is an index equivalent to the cross correlation coefficient for a given frequency domain. When calculating the coherence function, the two sets of brain activity time-series data in FIG. 4(a) are transformed by Fourier transformation, and the complex number acquired as a result is substituted into an expression defined as in illustrated in FIG. 4(d). Here, X(f) and Y(f) represent the Fourier transformation of the time series data x₁ and yᵢ, and X*(f) and Y*(f) represent the complex conjugate of X(f) and Y(f), and Coh represents the coherence function. The numerator as in FIG. 4(d) is the cross spectrum of xᵢ and yᵢ, and the denominator is the product of the auto spectrum of xᵢ and yᵢ. As illustrated in FIG. 4(c), the auto spectrum is the power spectrum of xᵢ and yᵢ. The coherence function is expressed as a frequency function, and so an arbitrary index has to be derived from the coherence function in order to quantify the degree of relationship between the two sets of time-series data. Examples include the average values or the integral values of specific frequency bands regarding the coherence function. Other indexes derived from the coherence function may be used as well.

### Other Examples of the Degree of Similarity

The degree of similarity according to the present embodiment may be any amount representing the degree of closeness from a dynamic perspective between the brain activity data for the two brain regions, and so is not limited to the cross correlation coefficient or the coherence function. For example, the difference between the time-average values of the two sets of brain activity time-series data (x₁, x₂, . . . , xₙ) and (y₁, y₂, . . . , yₙ) may be used as the degree of similarity. The difference between the time-average values of the two sets of brain activity time-series data is acquired by substituting values into the expression illustrated in FIG. 12B. As sensitivity of the signal detection elements and noise fluctuates spatially in the brain activity data, the absolute values of the signal at each measurement point also fluctuate. Here, normalization is performed when calculating the time-average values. For example, when a signal array (x₁, x₂, . . . , xₙ) is measured, the average value x′ for the signal array is subtracted from each signal value, and the signal array difference regarding the average value is calculated. Further, the start point of the signal array is standardized to zero, each signal value is divided by the start point x₁-x′, and then subtracted by one to normalize the signal array into a time-series array of the amount changed over time from the start point. FIG. 12B illustrates an expression to formulate the difference in the time-average values between the two signal arrays (x₁, x₂, . . . , xₙ) and (y₁, y₂, . . . , yₙ) in which the normalization procedure is added. This is an example of the degree of similarity in which a time-average value near zero represents similarity between the two sets of time-series data, and the farther the value is from zero, the similarity decreases. Additionally, any index that quantifies the degree of similarity between two sets of time-series data for some time period may be used such as the expression illustrated in FIG. 12C, in which the difference in the average rate of change for some arbitrary time period [a, b] (a<b) regarding the two sets of brain activity time-series data is calculated.

The smaller the stress is, the more the degree of similarity of the brain activity data for the multiple brain regions increases, and the difference between the time-average values or the difference in the average rate of change approaches zero. Conversely, the larger the stress, the more the degree of similarity of the brain activity data for the multiple brain regions decreases, and the more the difference between the time-average values of the difference in the average rate of change moves away from zero. Therefore, by indexing the degree of similarity between brain activity for multiple brain regions, information related to stress in subjects may be acquired.

According to the experiment implemented by the inventors, the corresponding relationship between the level of stress and the difference in the time-average values of low frequency components in particular, but also the difference in the average rates of change were discovered from the brain activity time-series data measured by an fMRI device. Therefore, it is beneficial to perform preprocessing to extract low frequency components using a low pass filter when calculating the difference between time average values or the difference in average rates of change regarding the brain activity time-series data related to changes in cerebral blood flow measured by an fMRI device.

### Setting Method for the Threshold

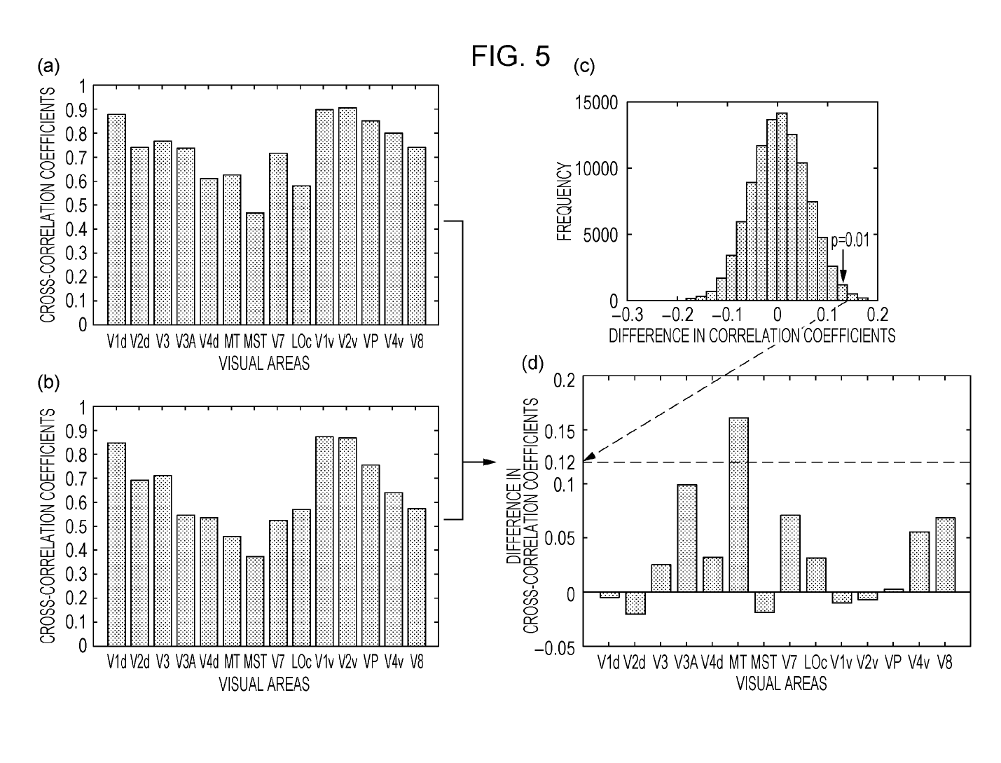

To set the threshold, it is preferable to apply the information acquiring method according to the present embodiment to previously selected subjects, and clearly establish the relationship between the level of stress and the cross correlation coefficient, for example. Specifically, a video A which does not provide stress and a video B which does provide stress is presented separately to the subject, and the brain activity of the subject is measured during the viewing of these videos. Then, how much stress the videos provided to the subjects, that is to say, the level of stress is quantified by implementing a psychophysical evaluation before and after the viewing of each video, the most-widely used of which is the Simulator Sickness Questionnaire (SSQ). Details on the SSQ are described in “Simulator sickness questionnaire: an enhanced method for quantifying simulator sickness”, Robert S. Kennedy, et al., International Journal of Aviation Psychology, 3 (3): 203-220 (1993). The brain activity correlation for when viewing video A and video B are calculated using the brain activity time-series data for the visual cortices. As an example, the graphs illustrated in the lower left of FIGS. 5(a) and 5(b) are bar charts illustrating the cross correlation coefficients calculated using the brain activity time-series data when the video A and the video B were presented. 14 visual areas are illustrated including the dorsal primary visual cortex (VW), the dorsal secondary visual cortex (V2d), the tertiary visual cortex (V3 and V3A), the dorsal fourth visual cortex (V4d), the middle temporal area (MT), the medial superior temporal area (MST), the seventh visual cortex (V7), the lateral occipital complex (LOc), the ventral primary visual cortex (V1v), the ventral secondary visual cortex (V2v), the ventral posterior area (VP), the ventral fourth visual cortex (V4v), and the eighth visual cortex (V8), and the vertical axis represents the size of each correlation value.

After implementing the SSQ before and after presenting video A and video B and confirming that video A does not provide stress and video B does provide stress, whether there is any difference in the brain activity correlation between the presentation of video A and video B is statistically tested to calculate the threshold for determining video that does not provide stress versus video that provide stress. FIG. 5(d) illustrates a bar chart illustrating the difference in the cross correlation coefficients illustrated in FIGS. 5(a) and 5(b). Among the differences in cross correlation coefficients illustrated in FIG. 5(d), two groups of cross correlation coefficients illustrated in FIGS. 5(a) and 5(b) are tested by a permutation test in order to determine statistically significant items. The permutation test is a non-parametric statistical significant difference test generally used when testing the presence of significant difference between samples of two groups. The procedure of the test is described below.

(i) A null hypothesis of “no difference between the cross correlation coefficients of two groups” is established.

(ii) Samples of the two groups of cross correlation coefficients are randomized

(iii) The randomized samples are again separated into two groups.

(iv) The difference in the average values of the samples of the two groups separated in step (iii) is calculated.

(v) Steps (ii) through (iv) are iterated until many samples of differences in the average values have been calculated.

(vi) The sample value coinciding with the level of significance is acquired from the distribution of the samples calculated in step (v).

(vii) The sample value acquired in step (vi) becomes the threshold used to reject the null hypothesis previously described.

FIG. 5(c) illustrates an example of the distribution (histogram) of the samples derived when performing the permutation test. As illustrated in FIG. 5(c), when performing calculations with the level of significance set to 1%, the difference between the two groups of brain activity correlations is roughly 0.12. That is to say, when the brain activity correlation between the left and right hemispheres for any of the 14 visual areas decreases by at least 0.12 over that illustrated in FIG. 5(a), the video being evaluated (hereafter, the video under evaluation) is determined to provide stress to the viewer. For example, regarding the brain activity correlation between the middle temporal areas (MTs) in the left and right hemispheres as illustrated in FIG. 5, the cross correlation coefficient calculated from the brain activity time-series data when video A was presented is 0.62 and so the threshold for determining whether the video under evaluation provides stress is the result of 0.62-0.12, which equals 0.50. Conversely, if there are no visual areas in which the amount of decrease is at least 0.12 for the brain activity correlation of the left and right hemispheres illustrated in FIG. 5, the video under evaluation is determined to not provide stress to the viewer. That described here is just one example, and any method may be used to clearly establish the correlation between the level of stress and the degree of similarity in the brain activity of the viewer, and then determine the threshold of the degree of similarity. Also, the example described uses the cross correlation coefficients of two sets of brain activity time-series data, but the threshold may be set by a similar procedure when using other types of degrees of similarity such as differences in time-average values or differences in average rates of change regarding two sets of brain activity time-series data.

### Information Acquiring Device

The information acquiring device according to the present embodiment acquires brain activity information, information related to stress for example, from biological subjects to which the sensory stimulus has been presented, and includes at least a stimulus presenting unit, a measuring unit, a calculating unit, and a measuring control unit. The stimulus presenting unit presents a sensory stimulus to at least one of the brain areas in biological subjects including the visual cortices of the cerebral cortex, the auditory cortices of the cerebral cortex, and the vestibular cortices of the cerebral cortex, and the measuring unit measures brain activity in multiple, different regions of the brain areas. The calculating unit calculates the degree of similarity in the brain activity data for the multiple regions, and the measuring control unit controls the measuring unit to measure the brain activity in the multiple regions of the brain areas when the sensory stimulus is presented by the stimulus presenting unit. The information acquiring device according to the present embodiment acquires the brain activity information (for example, information related to stress) by measuring the brain activity for the brain areas while the sensory stimulus is presented to the brain areas by the stimulus presenting unit.

The multiple regions in which brain activity is measured regarding the information acquiring device according to the present embodiment are preferably selected from the left and the right cerebral hemispheres.

The sensory stimulus regarding the information acquiring device according to the present embodiment is preferably a visual stimulus, and also is preferably a video. Also, the calculating unit regarding the information acquiring device according to the present embodiment preferably calculates at least one of cross correlation coefficient, coherence function, difference in time-average values, or the difference in average rates of change, as brain activity for the multiple measured regions, and acquires brain activity information, information related to stress for example, based on the degree of similarity.

Also, the measuring unit may use such devices as the previously described single neuron recording device, an electroencephalograph, a magnetoencephalograph, an fMRI device, a NIRS device, and a positron emission tomography device.

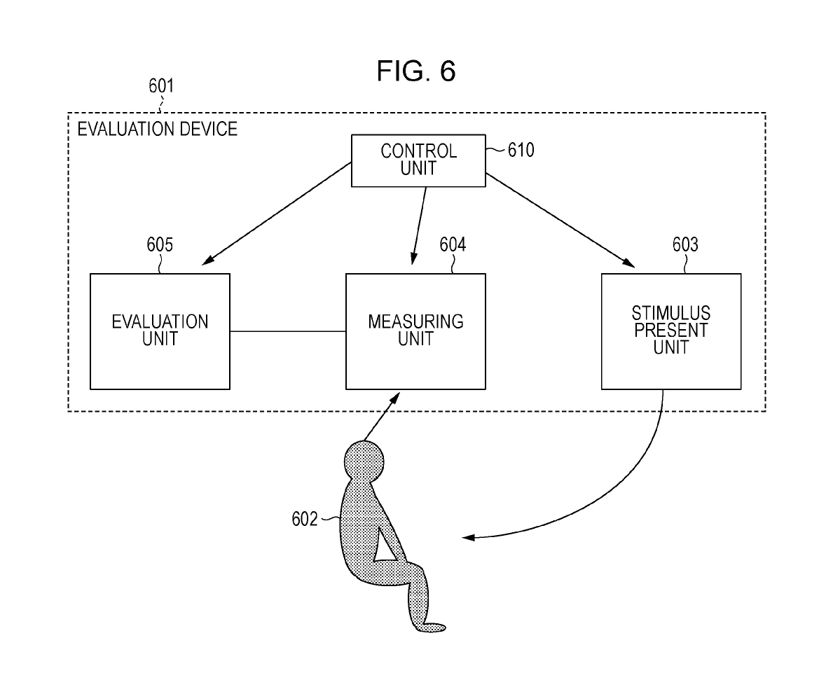

Next, as an example of the information acquiring device according to the present embodiment, an evaluation device to evaluate information related to stress will be described using FIG. 6.

An evaluation device 601 according to the present embodiment evaluates stress provided to subjects by the sensory stimulus. The evaluation device 601 includes at least a stimulus presenting unit 603 to present the stimulus to any of the brain areas including the visual cortices, the auditory cortices, and the vestibular cortices of a subject 602 by the sensory stimulus, a measuring unit 604 to measure brain activity for multiple of these brain areas while the stimulus is being presented to these areas, and an evaluation unit 605 to evaluate the stress based on the degree of similarity of brain activity measured by the measuring unit.

The measuring unit 604 measure brain activity when the stimulus is presented by the stimulus presenting unit 603. Also, the evaluation unit 605 evaluates stress based on the value of at least one value including the cross correlation coefficient, coherence function, difference in time-average values, or the difference in average rates of change calculated using the expression as in FIGS. 12A through 12D from the brain activity data measured by the measuring unit 604 before presenting stimulus with the stimulus presenting unit 603.

A specific example of the method to evaluate stress using the evaluation device according to the present embodiment will now be described. First, the sensory stimulus is presented using the stimulus presenting unit 603 to any brain area of the subject 602 including the visual cortices, auditory cortices, and vestibular cortices. The brain activity of multiple, different regions in the brain area is measured by the measuring unit 604 while the sensory stimulus is presented. Then, the evaluation unit 605 evaluates the stress based on the measured brain activity data, such as the degree of similarity.

The specific evaluation method may evaluate stress based on the value of at least one method including the cross correlation coefficient, coherence function, difference in time-average values, or the difference in average rates of change calculated from the brain activity data for the multiple brain areas selected from the brain areas to which the stimulus is presented by the stimulus presenting unit 603. The methods to calculate the cross correlation coefficient, coherence function, difference in time-average values, or the difference in average rates of change are as previously described.

Further, with a control unit 610, the stress evaluation device according to the present embodiment may control the stimulus presenting unit 603, the measuring unit 604, and the evaluation unit 605 for evaluating stress. For example, when a video is presented to the subject by the stimulus presenting unit 603, turning the presenting of the video on and off is controlled by the control unit 610. Also, the timing of measuring the brain activity may be automatically controlled by the control unit 610. Further, the evaluation device according to the present embodiment may be manually operated without using the previously described control unit 610.

Example of Evaluation Device

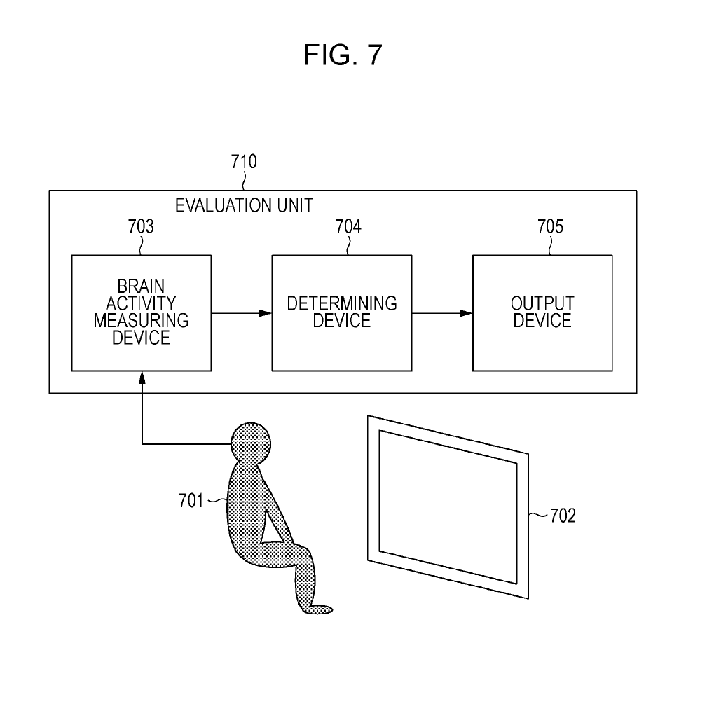

Next, a specific example of the evaluation device according to the present embodiment will be described using FIG. 7. Description will be made here regarding an example where the sensory stimulus is a video.

A viewer 701 watches a video under evaluation displayed by a display device 702. During this time, the brain activity in the visual cortices of the cerebral cortex of the viewer 701 is measured using a brain activity measuring device 703, which is the brain activity measuring method. The brain activity measuring device 703 may use the devices previously listed (Measuring Method for the Brain Activity). The previously described devices may measure brain activity in multiple regions of the cerebral cortex either separately or simultaneously. Among these, the fMRI device due to its high cost and large structure makes it difficult to implement for a large number of subjects, but it can measure brain activity in the specific brain region with a high level of sensitivity and spatial resolution, so it is preferable when highly accurate results from a small number of subjects is desirable. The positron emission tomography device shares the same high cost and large structure of the fMRI device also emits radioactive radiation, which limits applicability to a small number of humans acting as subjects, as well as the number of times any individual subject may be measured, but as this is applicable to measuring brain activity in specific brain regions with high accuracy, it is preferable when highly accurate results for a small number of subjects and measuring counts are desirable. With the electroencephalograph and NIRS device, spatial resolution is less than that of the fMRI device and positron emission tomography device, and so accuracy suffers, but signals corresponding to brain activity may be acquired by a simple configuration of attaching measuring sensors to various positions on the head of the subject, so it is preferable when testing a large number of subjects easily is desirable. At any rate, the brain positions measured according to the present invention are the visual cortices in the posterior regions, the auditory cortices in the temporal regions, and the vestibular cortices in the temporo-parietal regions in the brain, which limits the positions to be measured to the vicinity of these regions, so it is preferable to use a small number attached sensors.

The brain activity time-series data measured by the brain activity measuring device 703 is sent to a determining device 704. The determining device 704 is a general-purpose calculating function configured as a CPU and from a recording medium, in which a program for analyzing the measure brain activity time-series data is stored. The determining device 704 calculates the degree of similarity for two sets of brain activity time-series data using the brain activity time-series data from two different regions in the visual cortices of the cerebral cortex. The degree of similarity may be a correlation value regarding a time domain such as the cross correlation coefficient, or a correlation value regarding a frequency domain such as the coherence function. Also, as another example of the degree of similarity, the difference in time-average values or the difference in average rates of change relating to brain activity time-series data of the two regions may also be used.

The determination result from the determining device 704 is output to an output device 705. The combination of the brain activity measuring device 703, the determining device 704, and the output device 705 together form an evaluation unit 710.

The output device 705 sends signals to a display device such as a display connected to the determining device 704, displaying the determination result on a display screen and notifying the user. Also, signals may be sent to a printing device such as a printer connected to the determining device 704, printing the determination result on paper and notifying the user.

When the video under evaluation has a potential to cause 3D motion sickness, the output device 705 may be used as 3D video display device. Though not applicable to 3D video display devices using the auto stereoscopic method, when the 3D video display device using the anaglyph method or the polarization method, the viewer wears special glasses to view separate images for the left and the right eyes displayed on the screen. The images for left and the right eyes give binocular disparity, which is projected separately to the left and right eyes of the viewer. The viewer perceives a video with a sense of depth in response to the binocular disparity. Also, another example of a 3D video display device that displays images for the left and the right eyes with binocular disparity is a head mounted display (hereafter, HMD). In addition to the display of real video to the viewer in 3D, the HMD can also present an augmented reality or a mixed reality by augmenting and mixing the perspective of reality in the user by superimposing additional information such as CG into the video using a computer.

### Video Editing Method

A method to edit the content of the sensory stimulus using the brain activity information acquiring method such as the acquiring method of information related to stress will now be described. “Acquiring Method of Information Related to Stress” described that brain activity information such as information related to stress may be acquired by indexing the information related to stress provided to the subject by the sensory stimulus as a degree of similarity (cross correlation coefficient, coherence function, difference in time-average values, and difference in average rates of change) from the brain activity data for multiple brain regions. During the time that the sensory stimulus is presented, the index is calculated into time-series data so that the dynamic changes of the degree of similarity may be conceptualized, which enables the content of the sensory stimulus to be edited so that the appropriate level of stress is provided to the subject by the sensory stimulus.

For example, when the degree of similarity is the cross correlation coefficient is calculated from the brain activity data for the multiple brain regions, the time-series data of the cross correlation coefficient may be derived by sequentially calculating the cross correlation coefficient into the brain activity time-series data from the start point of the sensory stimulus. As previously described, when the cross correlation coefficient calculated from the brain activity data for the multiple brain regions significantly decreases, information may be acquired indicating that the sensory stimulus is providing noticeable stress to the subject. By referencing the time-series data regarding the cross correlation coefficient, the point in time where the cross correlation coefficient decreases below the threshold may be identified, and so information may be acquired indicating that content is included that provides noticeable stress to subjects in a temporal interval up to this point in time of the sensory stimulus. Therefore, the sensory stimulus may be edited so as to not provide excessive stress to the subject by editing a portion of the content of the sensory stimulus up to the point in time when the cross correlation coefficient decreases below the threshold, or by editing all of the sensory stimulus. Also, this does not mean to simply suppress the stress, but stimulate the subject with an appropriate amount of stress so as to arouse feelings of pleasant excitement and experience. For example, presenting the visual stimulus as a video with global visual motion information may provide an excessive stress that causes visually induced motion sickness in the subject, but may also be an excessive stress which produces a sensation of self-motion such that the subject feels as though they have been moving through the space within the video (vection) having an effect of arousing the feeling of presence. The cross correlation coefficient calculated from brain activity data for the multiple brain regions does not fall below the threshold, but by editing the content of the sensory stimulus so as to be asymptomatic, the sensory stimulus may be edited so that the stress provided to the subject is adjusted to an appropriate range.

When the index is the difference in time-average values or the difference in average rates of change regarding the brain activity data, the time-series data for the difference in time-average values or the difference in average rates of change may be derived in the same way as for the cross correlation coefficient by calculating a value at each point as the brain activity data is detected from the start point of the sensory stimulus. Therefore, by referencing the time-series data for the difference in time-average values or the difference in average rates of change, the content of the sensory stimulus may be edited so that the desired stress is provided to the subject.

### Editing Device

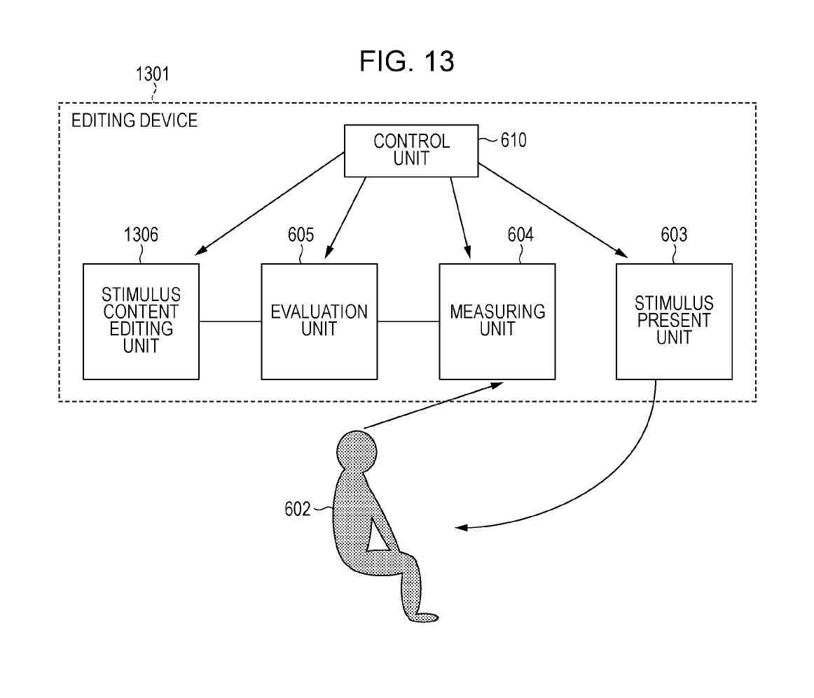

An editing device according to the present embodiment will be described with reference to FIG. 13. Numbers illustrated in FIG. 13 that are the same as those in FIG. 6 represent the same configuration components as described in the section “Evaluation Device.” An editing device 1301 is provisioned with a stimulus content editing unit 1306 for editing the content of the sensory stimulus presented to the subject in addition to the configuration components described in the section “Information Acquiring Device.” When the sensory stimulus is a visual stimulus, that is to say, a video, the stimulus content editing unit 1306 is configured with a computer provisioned with a video processing program capable of adjusting arbitrary parameters related to video effects such as the frame rate of the video or color correction, a video input device for photographing video, of a video photography equipment for adjusting the photographic environment of the video such as lighting and reflector boards. When the sensory stimulus is an auditory stimulus, the stimulus content editing unit 1306 is configured with a computer provisioned with an audio processing program to perform operations such as removing noise or integrating speech, an audio input device such as a microphone, or a speaker or similar for adjusting the volume. When the sensory stimulus is a sense of balance stimulus, the stimulus content editing unit 1306 is configured with arbitrary equipment to adjust an acceleration applied to the subject such as a suspension, and a computer provisioned with a program to control the behavior of this equipment.

Example of the Editing Device

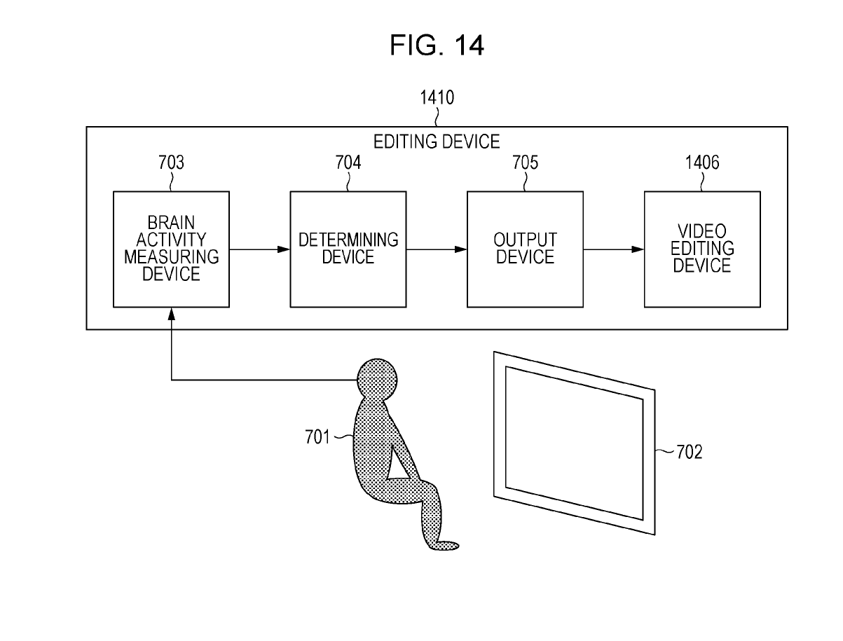

An example of the editing device according to the present embodiment will be described with reference to FIG. 14. Numbers illustrated in FIG. 14 that are the same as those in FIG. 7 represent the same configuration components as described in the section “Example of the Evaluation Device.” Similar to the section “Example of the Evaluation Device”, the example described assumes that the sensory stimulus is a visual stimulus, that is to say, a video, and that the brain activity information is information related to stress. The example of the editing device described here is provisioned with a video editing device 1406 for editing video in addition to the device disclosed in the section “Example of the Evaluation Device.” The determining device 704 calculates the changes over time of the degree of similarity, which is the index of the stress, based on brain activity time-series data from two different regions in the visual cortices of the cerebral cortex. The time-series data of the degree of similarity is output to the output device 705 where it is advertised to the user through output to a display device such as a display or a printing device such as a printer. The user then edits the video content presented to the subject based on the output result using the video editing device 1406, which includes video photography equipment such as lighting and reflection boards, video input devices such as a video camera, or a computer provisioned with a video processing program. When the sensory stimulus is an audio stimulus, an audio editing device configured of an audio input device and a computer provisioned with an audio processing program in place of the video editing device 1406. When the sensory stimulus is a sense of balance stimulus, an editing device for the acceleration present device configured of an arbitrary equipment to adjust an acceleration applied to the subject such as a suspension and an acceleration present device or a computer for controlling the behavior of this equipment in place of the video editing device 1406. The brain activity measuring device 703, the determining device 704, the output device 705, and one of either the video editing device 1406, the audio editing device, or the editing device for the acceleration present device combine to form an editing device 1410.

### Display Method

A method to present the sensory stimulus edited using the editing method will be described. Here, the sensory stimulus is assumed to a video. According to the section “Editing Method”, a process to edit the content of the video was described in which a time series array was calculated for the information related to stress in the subject based on the degree of similarity of the brain activity data for multiple, different regions while the video was presented, and by referencing the time series array regarding the information related to stress, the stress provided to the subject is adjusted to an appropriate range. The display method according to the present embodiment includes a process to present the edited video to the subject in addition to the process described in the section “Editing Method.” Specifically, a process is included to control the video edited using the stimulus content editing unit 1306 as described in the section “Editing Device” so as to be displayed by the stimulus presenting unit 603.

### Display Device

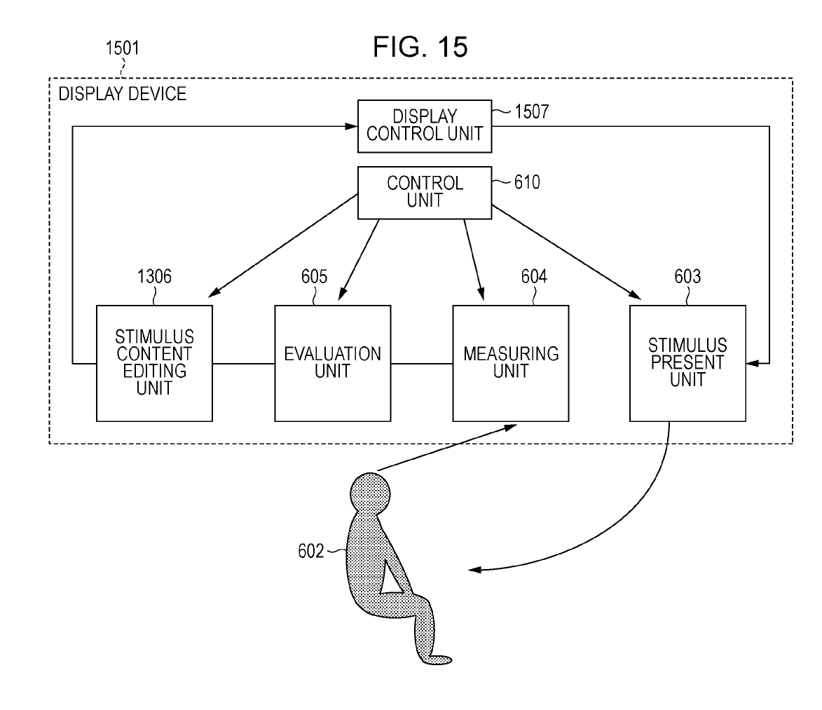

A display device according to the present invention will be described with reference to FIG. 15. Numbers illustrated in FIG. 15 that are the same as those in FIG. 13 represent the same configuration components as described in the section “Editing Device.” A display device 1501 includes a display control unit 1507 in addition to the configuration components described in the section “Editing Device.” The display control unit 1507 controls the display of the video by sending signals to the stimulus presenting unit 603 so as to display the video edited by the stimulus content editing unit 1306 to the subject. Further, the control unit 610 and the display control unit 1507 may be implemented as one device using the same calculating device.

Example of the Display Device

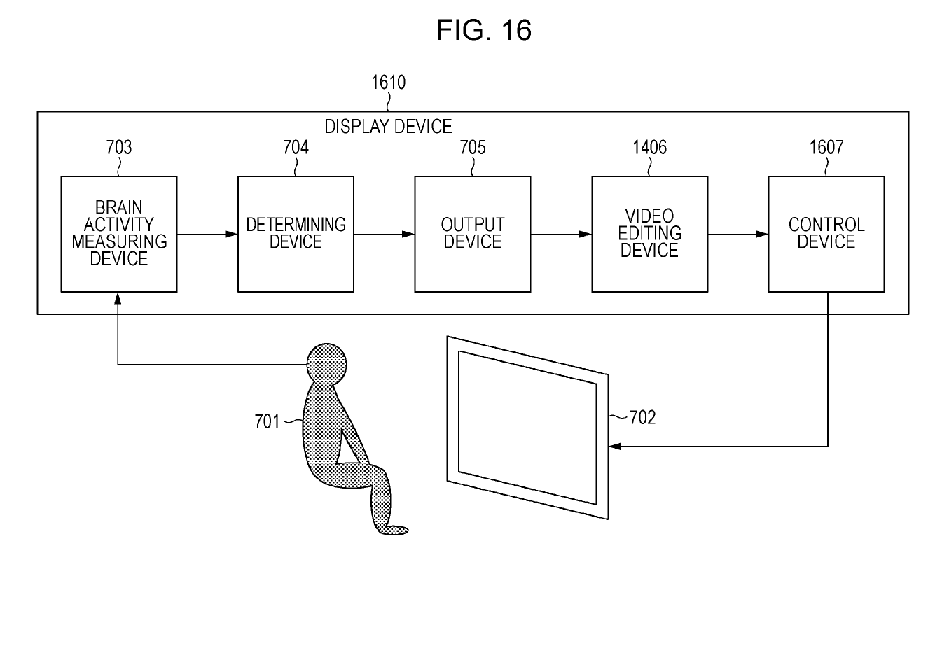

An example of the display device according to the present invention will be described with reference to FIG. 16. Numbers illustrated in FIG. 16 that are the same as those in FIG. 14 represent the same configuration components as described in the section “Example of the Editing Device.” The example of the display device described here is provisioned with a display control device 1607 to control the video edited by the video editing device 1406 so as to display to a display device such as a display, in addition to the device disclosed in the section “Example of the Editing Device.” The display control device 1607 does not only control the video edited by the video editing device 1406, but also may be configured to control the video to be evaluated (vide under evaluation). For example, let us assume that when performing a control to display the video under evaluation, the determining device 704 determines that the video under evaluation provides a noticeable stress to the subject 701. The determination result from the determining device 704 is sent to the video editing device 1406 by the output device 705, and then the video under evaluation is edited with the video editing device 1406. For example, the video editing device 1406 performs an edit to superimpose video information such as a text string functioning as a warning into the video under evaluation, and the display control device 1607 performs a control so that the edited video is displayed on a display device. The brain activity measuring device 703, the determining device 704, the output device 705, the video editing device 1406, and the display control device 1607 combine to form a display device 1610. That described here is just one example of the display control, and so is not limited thusly.

### Program

An example of the program according to the present embodiment is an information acquiring program configured to acquire brain activity information such as information related to stress from biological subjects to which the sensory stimulus is presented, and executes the processes including the processes (1) through (3) regarding the previously described information acquiring program.

Another example of the program according to the present embodiment is an information acquiring program configured to acquire brain activity information such as information related to stress from biological subjects to which the sensory stimulus is presented using the previously described information acquiring device to acquire the brain activity information. The information acquiring device includes a stimulus presenting unit for giving a sensory stimulus to at least one brain area of the biological subjects including the visual cortices of the cerebral cortex, the auditory cortices of the cerebral cortex, and the vestibular cortices of the cerebral cortex; a measuring unit to measure brain activity of multiple, different regions in the brain areas, a calculating unit to calculate the degree of similarity for the brain activity from the multiple regions, and a measuring control unit to control the measuring unit to measure the brain activity in the multiple regions in the brain areas when the sensory stimulus is presented by the stimulus presenting unit. The program in the present example is configured to use the information acquiring device to acquire the brain activity information such as information related to stress by executing the processes in which the brain activity is measured for multiple regions in the brain areas while the sensory stimulus is presented to the brain areas by the stimulus presenting unit is presented to at least one of the brain areas of biological subjects including the visual cortices of the cerebral cortex, the auditory cortices of the cerebral cortex, and the parieto-insular vestibular cortex of the cerebral cortex.

The program according to the present embodiment may be recorded on a recording medium, or may be downloaded from the Internet. The program is in a computer-readable format.

### Recording Medium

An example of the recording medium according to the present embodiment is a recording medium to which the information acquiring program configured to acquire brain activity information such as information related to stress from biological subjects to which the sensory stimulus is presented is stored, and records the information acquiring program that executes the processes including the previously described processes (1) through (3) in a computer-readable format.

Another example of the recording medium according to the present embodiment is a recording medium that records the information acquiring program configured to acquire brain activity information such as information related to stress from biological subjects to which the sensory stimulus is presented using the previously described information acquiring device to acquire the brain activity information. The information acquiring device includes a stimulus presenting unit for presenting a sensory stimulus to at least one brain area of the biological subjects including the visual cortices of the cerebral cortex, the auditory cortices of the cerebral cortex, and the vestibular cortices of the cerebral cortex; a measuring unit to measure brain activity of multiple, different regions in the brain areas, a calculating unit to calculate the degree of similarity for the brain activity from the multiple regions, and a measuring control unit to control the measuring unit to measure the brain activity in the multiple regions in the brain areas when the sensory stimulus is presented by the stimulus presenting unit. The recording medium in the present example records in a computer-readable format the information acquiring program configured to use the information acquiring device to acquire the brain activity information such as information related to stress by executing the processes in which the brain activity is measured for multiple regions in the brain areas while the sensory stimulus is presented to the brain areas by the stimulus presenting unit is presented to at least one of the brain areas of biological subjects including the visual cortices of the cerebral cortex, the auditory cortices of the cerebral cortex, and the vestibular cortices of the cerebral cortex.

The recording medium may be a CD (CDR, CDRW, etc.), a DVD (DVDR, DVDRW, etc.), a flash memory, a hard disk, a magnetic tape, or a floppy disk.

### EXAMPLES

### Example 1

The present embodiment will be described using the device described in the section “Example of the Evaluation Device.” Brain activity in the middle temporal area (MT) in both the right and left hemisphere of the viewer is measured, the cross correlation coefficient is calculated using the brain activity data, and whether the video under evaluation causes visually induced motion sickness is determined.

The brain activity measuring device used according to the present embodiment is an fMRI device. The display device is configured of a screen provisioned within the bore of the fMRI device, and a video projector arranged externally from the measuring unit to project the video on the screen. The video projector is controlled by a control computer, and performs the display and non-display of the video under evaluation, the display of the determination results, etc.

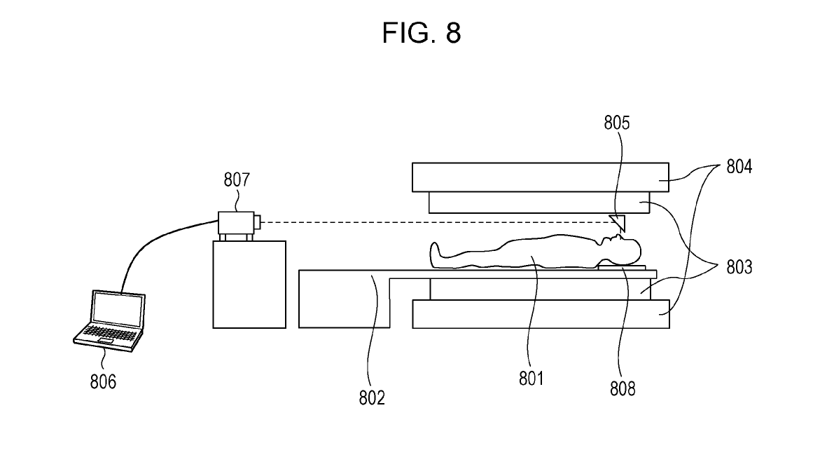

FIG. 8 is a cross-sectional diagram of the fMRI device according to the present example. A viewer 801 lies on a bed 802 attached to the fMRI device, and a gradient magnetic coil 803 and a superconducting magnet 804 are arranged within the measuring unit. A screen 805 is provisioned near the eyes of the viewer 801 in the measuring unit, and a control computer 806 controls a video projector 807 to project the video from outside the measuring unit. A signal detection coil 808 is arranged near the occipital area of viewer, and electromagnetic signals correlating to changes in the cerebral blood flow produced from changes in action potentials in neurons are detected.

According to the present example, evaluation is performed on video including scenes which present a feeling shakiness, and configured so as to potentially cause visually induced motion sickness in the viewer 801 by watching the video. Brain activity was measured particularly for the visual cortices distributed in the occipital area of the left and right cerebral hemispheres.

The brain activity time-series data measured by the fMRI device is sent to and recorded in the determining device, and then analyzed by the internal program. The brain activity time-series data includes noise from the fMRI device and noise from biological activity such as the heart beating and breathing. Therefore, it is preferable to perform a preprocessing to remove the components other than the brain activity response to the video.

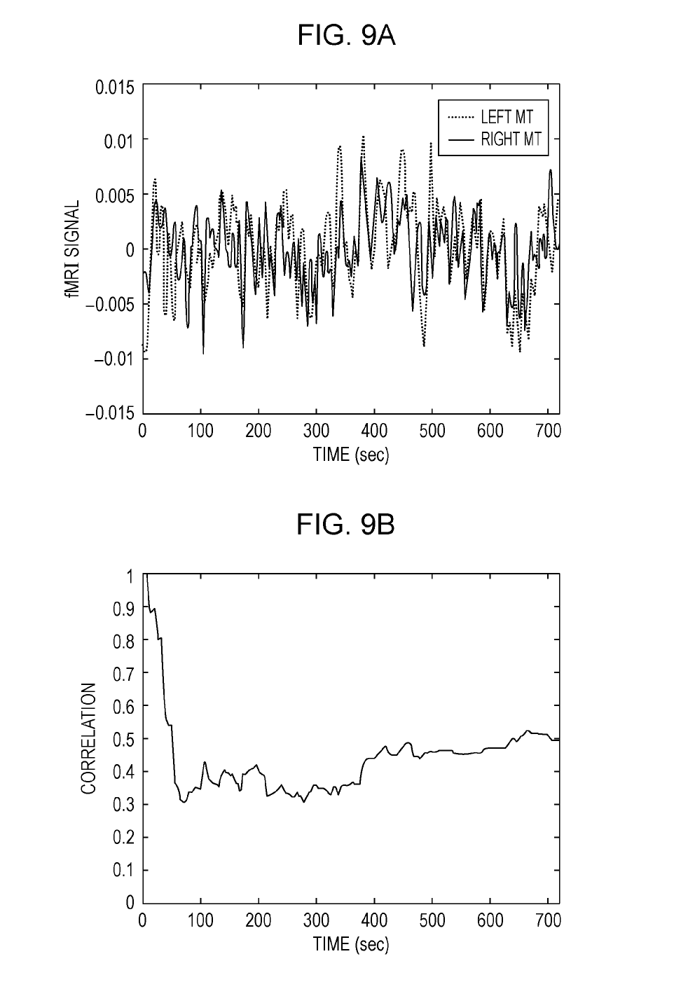

FIG. 9A illustrates the brain activity time-series data regarding the middle temporal area (MT) of the left and right hemispheres after executing the preprocessing. The cross correlation coefficient is calculated using the brain activity time-series data regarding the middle temporal area (MT) in the left hemisphere and the brain activity time-series data regarding the middle temporal area (MT) in the right hemisphere as illustrated in FIG. 9A. As a result, the cross correlation coefficient is 0.46.

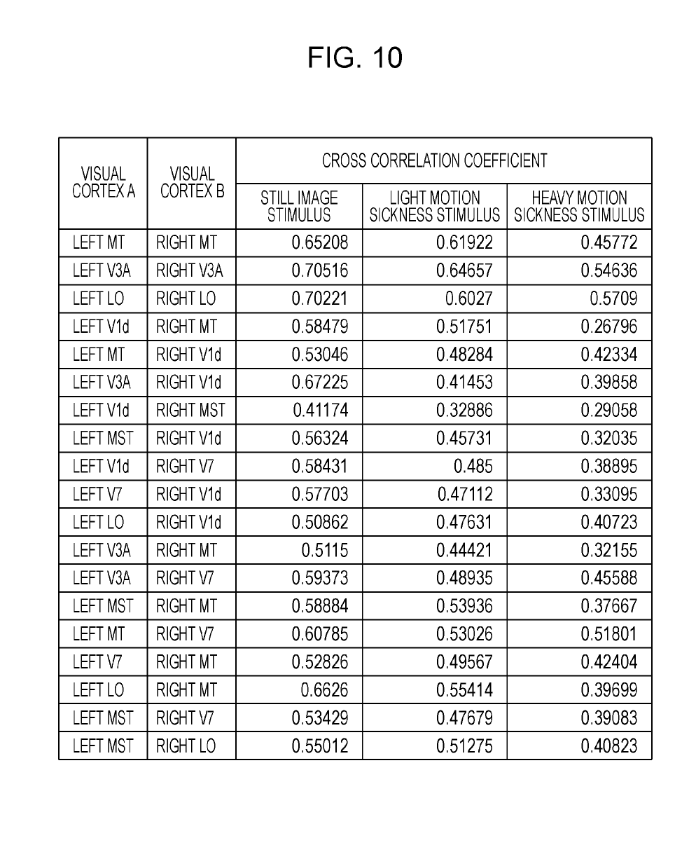

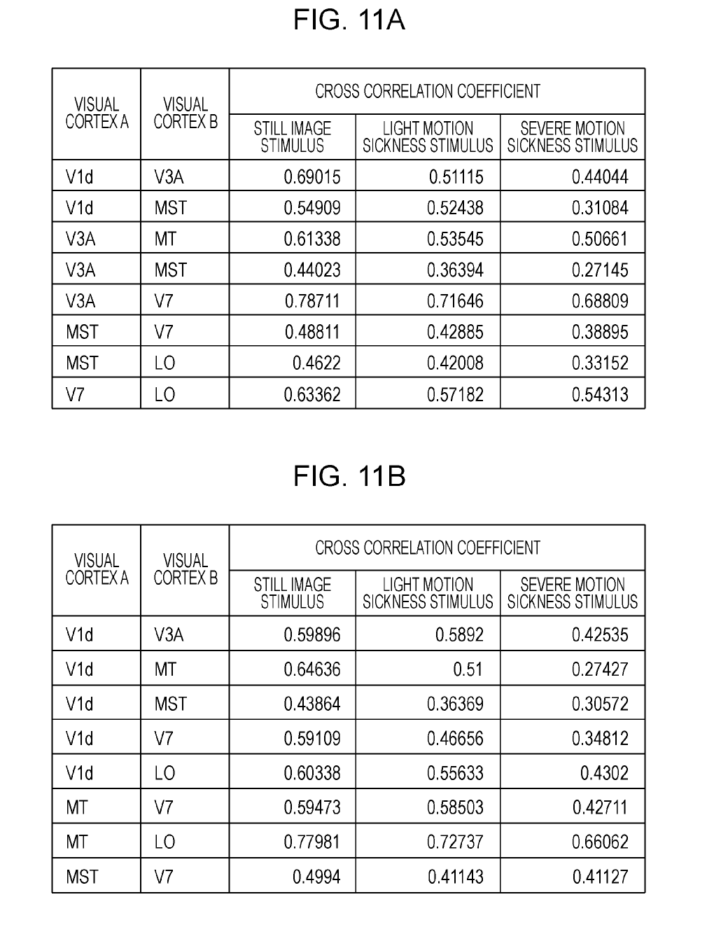

The cross correlation coefficient is calculated similarly regarding brain activity time-series data from the various brain areas. FIGS. 10, 11A, and 11B illustrate such results. FIG. 10 is a collection of the cross correlation coefficients calculated by the expression illustrated in FIG. 12A when brain activity was measured from one brain area was selected from the visual cortices in the left cerebral hemisphere and one brain area was selected from the visual cortices in the right cerebral hemisphere. In FIG. 10, left indicates the left cerebral hemisphere, and right indicates the right cerebral hemisphere. For example, the right MT represents the middle temporal area (MT) in the right cerebral hemisphere.

FIGS. 11A and 11B are collections of the cross correlation coefficients calculated by the expression illustrated in FIG. 12A when brain activity was measured from two different brain areas (visual cortex A and visual cortex B) were selected from both the visual cortices in the left cerebral hemisphere and the right cerebral hemisphere.

As illustrated in FIGS. 10, 11A, and 11B, as compared to when a still image not providing a stress (denoted as still image stimulus) is presented, when the video providing a light stress (denoted as light motion sickness stimulus) is presented or a video providing a heavy stress (denoted as heavy motion sickness stimulus) was presented, it was found that as the cross correlation coefficient calculated from the brain activity time-series data from the brain areas in both hemispheres decreased, the stress may be evaluated as increasing.

The cross correlation coefficient calculated as previously described is compared to a previously set threshold. If the cross correlation coefficient is larger than the threshold, the determining device sends to the output device a determination result indicating that the video under evaluation does not provide stress such as visually induced motion sickness to the viewer. Also, if the cross correlation coefficient is smaller than the threshold, the determining device sends to the output device a determination result indicating that the video under evaluation does provide stress such as visually induced motion sickness to the viewer.

The threshold may be set to an arbitrary value depending on the level of strictness in determinations desired by the user. Typically, the present invention is applied to a predetermined arbitrary subject as previously described, and so a reference value for brain activity correlations enabling determination of stress provided to the viewer by the video should be determined. In this case, by increasing the number of subjects to improve statistical accuracy, a more universal threshold may be set.

Conversely, a reference video different from the video under valuation known to provide stress to the viewer may be prepared, and then the threshold may be set to a reference value of brain activity correlations in viewers to which have been presented the reference video. Individuals behave differently to stress caused by visually induced motion sickness and 3D motion sickness, and also physical conditions on the day of testing influence these behaviors. Therefore, calculating the brain activity correlations in viewers against the reference video together with evaluating the video under evaluation to set the threshold using this brain activity correlation as a reference value helps prevent the influence of individual differences and differences in daily physical conditions to appear in the brain activity information to be acquired.

The user is notified of the determination result sent from the determining device by the output device. For example, signals are sent to a display connected to the determining device so as to display the determination result. Also, signals may be sent to a printer connected to the determining device so as to print the determination result.

As the brain activity measuring device used according to the present embodiment is an fMRI device, the brain activity in various regions in the visual cortices of the cerebral cortex are measured at a high spatial resolution, which enables a highly accurate evaluation result to be acquired. For example, the present example is preferable when measuring the brain activity for a specific brain area within the visual cortices of the cerebral cortex such as the middle temporal area (MT).

### Example 2

As another example different from the (example 1) above, the present embodiment will be described using an example in which the fMRI device is replaced by an electroencephalograph. Brain activity is measured near the middle temporal area (MT) in the left and right hemispheres of the viewer, the coherence function is calculated using the brain activity data, and whether the video under evaluation causes visually induced motion sickness is determined Reference electrodes are attached to the earlobes, and active electrodes are arranged on positions T₅ and T₆ in the posterior temporal regions nearest to the middle temporal area (MT) in compliance with the International 10-20 System.

The visual distance of the video under evaluation is adjusted to fit the screen size so that the visual angle is aligned to that of the (example 1) above, and then displayed on a commercial display. Two types of video are used in the present example. A first video (hereafter, video 1) was a 6 minutes of movie that was capable of providing heavy visually induced motion sickness. A second video (hereafter, video 2) was a 6 minutes of movie that was capable of providing light visually induced motion sickness. According to the present example, video 2 was presented to the viewer first, and then after a 5-minute rest period, video 2 was presented.

The brain wave data measured while video 1 and video 2 were presented was substituted in the expression illustrated in FIG. 12D to calculate a mean-square coherence function. The mean-square coherence function is a number value acquired by squaring the average value of the coherence function over some frequency bands, and is represented as a value between zero to one. As the mean-square coherence function approaches one, two signals are thought to be similar, and as it approaches zero, they are thought to be different.

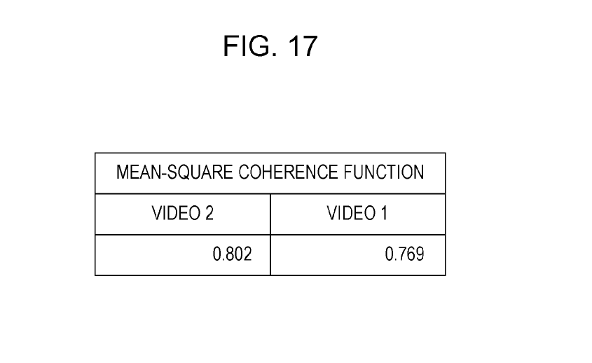

FIG. 17 illustrates the value of the mean-square coherence function calculated from the brain wave data acquired by the present example. Video 1 and video 2 in FIG. 17 represent the mean-square coherence function calculated from the brain wave data when video 1 and video 2 were presented. As may be understood from subtracting the values of the mean-square coherence function (0.802-0.769=0.033), the value of video 1 is 0.033 less than the value of video 2. That is to say, the presenting of video 1 caused visually induced motion sickness in the viewer, which disturbed the temporal pattern of the brain activity near the left and right middle temporal area (MT), and as a result, this led to the decrease in the value of the mean, and as a result, this led to the decrease in the value of the mean-square coherence function.

A threshold may be arbitrarily set to the desired strictness of the user to determine whether the change in the mean-square coherence function is abnormal, that is to say, whether the visually induced motion sickness in the viewer caused by the video deviates from a limit. The threshold may also be set using a statistical method such as the permutation test previously described.

As previously described, the electroencephalograph is less expensive and less restrictive than the fMRI device, and so is preferable in many situations. It is particularly preferable in implementing a system in which the above-mentioned procedures can be executed automatically and in real time. For example, by implementing such a system in the HMD, the video may be evaluated, edited, and displayed in real time as the HMD is in use. The editing and display of the video will be described specifically in the following example 3 and example 4.

### Example 3

The present example will be described using the device expressed in the previously described section “Example of the Editing Device.” In this example, brain activity for the middle temporal area (MT) in the left and right hemispheres of the viewer are measured, the cross correlation coefficient is calculated as time-series data using the brain activity data, points in time where the video under evaluation caused visually induced motion sickness in the viewer are identified, and the content of the video under evaluation is edited. FIG. 9A illustrates brain activity time-series data for the middle temporal area (MT) in the left and right hemispheres when a 12-minute video was presented to the subject, and FIG. 9B illustrates the time-series data for the cross correlation coefficient calculated from the brain activity time-series data. The time-series data for the cross correlation coefficient illustrated in FIG. 9B is sequentially calculated using the brain activity time-series data from the start point of the video at each point as the brain activity data was detected. If the threshold statistically estimated as in the section “Threshold Setting Method” of 0.50 is used as a target, the time-series data for the cross correlation coefficient in FIG. 9B illustrates that at about 50 seconds from the start of the video, the value falls below the threshold, and this indicates that a statistically significant stress was provided to the subject by the video content up to this point in time. That is to say, in order to create a suitable stress provided to the subject that does not fall below the threshold, it may be determined that the content at least from the start of the video until to 50-second mark has to be edited. Also, the cross correlation coefficient falls below the threshold for time after 50 seconds from the start of the video. Therefore, it may be determined that editing is preferable as content continually providing a statistically significant stress is included in the presented video.

The stress provided to the subject may be controlled through the feeling of shakiness by editing the amount of visual motion included in scenes where physical objects move in the video, or the background moves on a global scale, for example. Specifically, editing is performed using the video editing device 1406 such as replacing a portion of the video content from the start of the video up to the 50-second mark with video that has little visual motion information or is more unlikely to provide stress to the subject so as to suppress the feeling of shakiness perceived by the subject. In this case, it is preferable to calculate an index using brain activity data for multiple regions in the middle temporal area (MT) in the left and right hemispheres or the medial superior temporal area (MST) in the left and right hemispheres as many neurons that selectively respond to visual motion exist there.

Also, when the video under evaluation is a 3D video, the stress provided to the subject may be controlled through the stereoscopic effect by editing the amount of depth included in scenes such as the binocular disparity. Specifically, video processing is performed using the video editing device 1406 such as replacing the video projected on the left and right eyes with video that has less binocular disparity. In this case, it is preferable to calculate an index using brain activity data for multiple regions in the tertiary visual cortices (V3 and V3A), the middle temporal area (MT), the medial superior temporal area (MST), and the lateral occipital complex (LOc) in the left and right hemispheres as many neurons that selectively respond to binocular disparity exist there.

### Example 4

The present example will be described using the device expressed in the section “Example of the Display Device.” Similar to the (example 3) above, brain activity for the middle temporal area (MT) in the left and right hemispheres of the viewer is used to calculate the cross correlation coefficient as time-series data. The time-series data for the cross correlation coefficient is referenced to identify points in time where the video under evaluation caused visually induced motion sickness in the viewer, and the content of the video under evaluation is edited. For example, the time-series data for the cross correlation coefficient in FIG. 9B illustrates that at about 50 seconds from the start of the video, the value falls below the threshold, and this indicates that a statistically significant stress was provided to the subject by the video content up to this point in time. At this point, editing is performed such as superimposing video information such as a text string and icon into the video for at least the 50 seconds from the start of the video that cautions the viewer with a statement such as “Continuing to watch this video may result in the occurrence of visually induced motion sickness”, and then the edited video is controlled so as to be displayed to the viewer. By implementing this kind of display control, the provision of an excessive stress to the viewer may be prevented.

The display device in the present example may be implemented as the HMD equipped with a less-restrictive brain activity measuring device such as an electroencephalograph or a NIRS device. The HMD may be used in an environment provisioned with a calculating device for performing high speed video processing in addition to the display device in order to not only photograph and display video in the actual environment of the user, but to also superimpose desired video information such as CG. The previously described display device may be implemented by installing a program for performing video processing, and a program for analyzing brain activity data measured by the brain activity measuring device into the calculating device.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-091278, filed Apr. 12, 2012 and No. 2012-272671, filed Dec. 13, 2012, which are hereby incorporated by reference herein in their entirety.

1. An information acquiring method to acquire brain activity information from biological subjects presented a sensory stimulus, the method comprising:
(1) a process to present the sensory stimulus to at least one brain area of the biological subjects including the visual cortices of the cerebral cortex, the auditory cortices of the cerebral cortex, and the vestibular cortices of the cerebral cortex;
(2) a process to measure brain activity in multiple, different regions of the brain areas while the sensory stimulus is presented; and
(3) a process to acquire the brain activity information on the basis of the brain activity data for the multiple regions.

2. The information acquiring method according to claim 1, wherein the multiple regions are selected from the left hemisphere and the right hemisphere of the cerebral cortex.

3. The information acquiring method according to claim 1, wherein the sensory stimulus is at least one type of stimulus including a visual stimulus and a sense of balance stimulus.

4. The information acquiring method according to claim 1, wherein the sensory stimulus is a video.

5. The information acquiring method according to claim 1, wherein the brain activity information is information related to stress in the biological subjects.

6. The information acquiring method according to claim 1, wherein the process (3) acquires brain activity information based on at least one method including a cross correlation coefficient, difference in time-average values, difference in average rates of change, and a coherence function from the brain activity data for the multiple regions measured in process (2).

7. The information acquiring method according to claim 1, wherein video is evaluated on the basis of the brain activity information.

8. A video editing method comprising the information acquiring method according to claim 1, wherein content of the sensory stimulus is edited on the basis of time-series of the brain activity information.

9. A video display method comprising the editing method according to claim 8, further comprising:
a process of the edited video being displayed to the biological subject.

10. An information acquiring device to acquire brain activity information from biological subjects presented a sensory stimulus, the device comprising:
a stimulus presenting unit configured to present a sensory stimulus which changes over time to at least one brain area of the biological subjects including the visual cortices of the cerebral cortex, the auditory cortices of the cerebral cortex, and the vestibular cortices of the cerebral cortex;
a measuring unit configured to measure brain activity in multiple, different regions of the brain areas; and
a measuring control unit configured to control the measuring unit to measure the brain activity in the multiple regions while the sensory stimulus is presented to the brain areas by the stimulus presenting unit; wherein
the brain activity information is acquired by measuring the brain activity in the multiple regions while the sensory stimulus is presented to the brain areas.

11. The information acquiring device according to claim 10, wherein the multiple regions are selected from the left hemisphere and the right hemisphere of the cerebral cortex.

12. The information acquiring device according to claim 10, wherein the sensory stimulus is at least one type of stimulus including a visual stimulus and a sense of balance stimulus.

13. The information acquiring device according to claim 10, wherein the sensory stimulus is a video.

14. The information acquiring device according to claim 10, wherein the brain activity information is information related to stress in the biological subjects.

15. The information acquiring device according to claim 10, wherein brain activity information is acquired on the basis of a cross correlation coefficient from the brain activity for the measured multiple regions.

16. An evaluation device comprising:
the information acquiring device according to claim 10;
wherein video is evaluated on the basis of the brain activity information.

17. An editing device comprising:
the information acquiring device according to claim 10; and
a stimulus content editing unit configured to edit content of the sensory stimulus, based on time-series data regarding the brain activity information.

18. A video display device comprising:
the editing device according to claim 17; and
a display control unit configured to display edited video to the biological subject.

19. An information acquiring program to acquire brain activity information from biological subjects presented a sensory stimulus by executing the following processes:
(1) a process to present the sensory stimulus to at least one brain area of the biological subjects including the visual cortices of the cerebral cortex, the auditory cortices of the cerebral cortex, and the vestibular cortices of the cerebral cortex;
(2) a process to measure brain activity in multiple, different regions of the brain areas while the sensory stimulus is presented; and
(3) a process to acquire the brain activity information on the basis of the brain activity data for the multiple regions.

20. A computer-readable recording medium in which is recorded an information acquiring program to acquire brain activity information from biological subjects presented with a sensory stimulus, the information acquiring program executing the following processes:
(1) a process to present the sensory stimulus to at least one brain area of the biological subjects including the visual cortices of the cerebral cortex, the auditory cortices of the cerebral cortex, and the vestibular cortices of the cerebral cortex;
(2) a process to measure brain activity in multiple, different regions of the brain areas while the sensory stimulus is presented; and
(3) a process to acquire the brain activity information on the basis of the brain activity data for the multiple regions.

21. An information acquiring program to execute the processes to acquire brain activity information by measuring the brain activity in multiple regions while the sensory stimulus is presented to the brain areas, using an information acquiring device which includes
a stimulus presenting unit configured to present a sensory stimulus to at least one brain area of the biological subjects including the visual cortices of the cerebral cortex, the auditory cortices of the cerebral cortex, and the vestibular cortices of the cerebral cortex,
a measuring unit configured to measure brain activity in multiple, different regions of the brain areas, and
a measuring control unit configured to control the measuring unit to measure the brain activity in the multiple regions while the sensory stimulus is presented to the brain areas by the stimulus presenting unit.

22. A computer-readable recording medium to record an information acquiring program, in which the information acquiring program executes processes to acquire brain activity by measuring the brain activity information in the multiple regions while the sensory stimulus is presented to the brain areas, using an information acquiring device which includes
a stimulus presenting unit configured to present a sensory stimulus which changes over time to at least one brain area of the biological subjects including the visual cortices of the cerebral cortex, the auditory cortices of the cerebral cortex, and the vestibular cortices of the cerebral cortex,
a measuring unit configured to measure brain activity in multiple, different regions of the brain areas, and
a measuring control unit configured to control the measuring unit to measure the brain activity in the multiple regions while the sensory stimulus is presented to the brain areas by the stimulus presenting unit.
